(19) United States
(12) Patent Application Publication
(10) Pub. No.: US 2005/0074468 A1
(43) Pub. Date: Apr. 7, 2005

(54) Topical compositions containing nonimmunosuppressive cyclosporin derivatives for treating hair loss

(76) Inventors: Sang-Nyun Kim, Daejeon (KR); Ho-Jeong Ahn, Daejeon (KR); Chang-Woo Lee, Daejeon (KR); Min-Ho Lee, Daejeon (KR); Jung-Hun Kim, Daejeon (KR); Jong-Ii Kim, Daejeon (KR); Seung-Jin Kim, Seoul (KR); Ho-Song Cho, Daejeon (KR); Heon-Sik Lee, Daejeon (KR); Hyung-Jin Kim, Daejeon (KR); Jin-Chul Kim, Daejeon (KR); Seung-Kyu Park, Daejeon (KR)

Correspondence Address: VENABLE, BAETJER, HOWARD AND CIVILETTI, LLP, P.O. BOX 34385, WASHINGTON, DC, 20043-9998, US

(21) Appl. No.: 10/477,268

(22) PCT Filed: May 9, 2002
(86) PCT No.: PCT/KR02/00861

(30) Foreign Application Priority Data
May 15, 2001 (KR) 2001/26503

(51) Int. Cl. A61K 9/00

(52) U.S. Cl. 424/400

(57) ABSTRACT

The present invention discloses a topical scalp and transdermal preparation with excellent penetration to the skin and follicle, containing a [γ-hydroxy-N-methyl-L-leucine⁴] cylosporin derivative which is a non-immunosuppressive component with hair growth stimulating ability. The topical scalp and transdermal preparation is prepared by incorporating the cyclosporin derivative into a liposome, microcapsule, micro-sphere, composite particle or emulsion, capable of being employed as a hair growth stimulating agent and applied for the prevention of hair loss.

### TECHNICAL FIELD

The present invention discloses a topical scalp and transdermal preparation with excellent penetration to the skin and follicle, containing a [γ-hydroxy-N-methyl-L-leucine⁴] cylosporin derivative which has a hair restoring ability with no immunosuppressive activity. The preparation is characterized in that the cyclosporin derivative is encapsulated in a liposome, microcapsule, microsphere, composite particle or emulsion, capable of being employed as a hair restoring agent and applied for the prevention of hair loss.

### BACKGROUND ART

On average, the human scalp contains about 100,000 to 150,000 hairs. Each hair has three main stages of growth: anagen, catagen and telogen, after which the hair falls out. This hair growth cycle is repetitive and the duration of one cycle is different from other cycles, ranging from approximately 3 to 6 years. Thus, the average adult normally loses about 50 to 100 hairs every day. In general, alopecia refers to a phenomenon wherein duration of the anagen growth phase is shortened and the percentage of hairs in the catagen and telogen phases increases, whereby the number of lost hairs is abnormally increased.

There are many theories to explain loss of hair, including for example, poor blood circulation, excessive functioning of male sex hormone, excessive production and secretion of sebum, deterioration of scalp by peroxides, bacteria, etc., hereditary factors, aging, stress, etc. However, explicit mechanisms have not been revealed. Recently, the population suffering from hair loss is tending to increase, since changing dietary habits and stress imposed on individuals due to modern social environments, etc. have increased. Also, the age of the individuals affected by alopecia is dropping and furthermore, the population of female alopecia sufferers is rising.

One of preparations which are most commonly used for treatment and prevention of alopecia is one that contains minoxidil. There are two hair-regrowth agents which have received approval from the U.S. Food and Drug Administration, and minoxidil is one of those approved hair-regrowth agents. Minoxidil was originally developed as a hypertension drug for the purpose of reducing blood pressure. However, when using this drug, as a side effect, a trichogenous effect was observed and thereafter, this drug became famous as a hair-regrowth agent. Although mechanisms by which minoxidil works as a hair-regrowth agent is not clearly understood, it is inferred that minoxidil increases blood flow by expansion of blood vessels, whereby roots of hairs are supplied with more nutrition and eventually, growth of hairs are promoted.

Such a model of blood flow increase has been indirectly supported by a recent report that minoxidil enhances the expression of vascular endothelial growth factor (VEGF), a growth factor associated with vasodilatation in the dermal papilla which is a main cell making up the hair roots (Br. J. of Dermatol., 1998, 138:407-411). Also, other than the vasodilative effect of the minoxidil in the hair-restoring mechanism, it has been reported that minoxidil promotes activation of dermal papilla cells in the roots of hair incubated in vitro, and growth of hair follicles in a tissue culture of follicles in vitro (Skin Pharmacol., 1996, 9:3-8 and J. Invest. Dermatol., 1989, 92:315-320). These facts indicate that minoxidil may work directly on the roots of hair as a growth factor.

In addition, finasteride, a main component of Propecia which has started to be sold by Merck, is used for treatment of alopecia. It inhibits conversion of the male hormone testosterone into dihydrotestosterone, which is a more potent male hormone than testosterone. On December of 1997, the 1 mg finasteride tablet was approved by the US FDA as a hair-regrowth agent for treatment of male pattern hair loss in men only, and is now commercially available. In clinical studies, it has been demonstrated to have a significant trichogenous effect. However, there has been a report that finasteride may inhibit male sexual function as a side effect (J. Am. Acad Dermatol., 1998, 39:578-589). Since neither finasteride nor minoxidil show superior effect in clinical tests, and there is concern about side effects, many researches are conducted to develop a new and improved hair-regrowth agents.

The cyclosporin family of drugs has immunosuppressive activity. It is also effective to inhibit growth of virus, fungus, protozoan, etc. and has various physiological effects such as nephrotoxicity, hepatotoxicity, hypertension, enlargement of periodontium, trichogenous effect, and so on, as side effects (Advances in Pharmacol., 1996, 35:114-246 and Drug Safety, 1994, 10:310-317). Cyclosporin A, a representative cyclosporin, is a cyclic peptide having the following Chemical Formula, which comprises 11 amino acids, including several N-methyl amino acids and D-alanine at No. 8 residue. 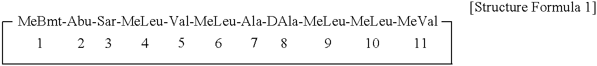
where MeBrnt is N-methyl-(4R)-4-[(E)-2-butenyl]-4-methyl-L-threonine, Abu is L-α-aminobutyric acid, Sar is sarcosine, MeLeu is N-methyl-L-leucine, Val is L-valine, Ala is L-alanine, DAla is D-alanine, and MeVal is N-methyl-L-valine.

The amino acid form of cyclosporin A of the above Chemical Formula 1 is L-configuration, unless otherwise specified. The residue numbering of amino acids starts from MeBmt and proceeds clockwise, i.e. 1 for MeBmt and 11 for the last MeVal (N-methyl-L-valine) as shown in the Structure Formula 1. Nomenclature of various derivatives including cyclosporin derivatives A to Z, follows methods commonly used (Helv. Chim. Acta, 1987, 70:13-36). For example, if Abu, the No. 2 residue of cyclosporin A is substituted with L-alanine, L-threonine, L-valine or L-norvaline, the derivatives thus prepared are named cyclosporin B, cyclosporin C, cyclosporin D or cyclosporin G, respectively. Further, when the amino acid residues of the cyclosporin derivatives differ from those of cyclosporin A, the derivatives are named by describing the substituent.

For example, if sarcosine, the No. 3 residue of cyclosporin A, is substituted with N-methyl-D-Abu³ and N-methyl-D-Nva³, the derivatives thus prepared are named [N-methyl-D-Abu³] cyclosporin A and [N-methyl-D-Nva³] cyclosporin A, respectively.

Similarly, when a hydroxyl group is added to the gamma carbon in N-methyl-L-leucine, the residue No. 4 of cyclosporin, by microbial metabolism, being substituted to [γ-hydroxy-N-methyl-L-leucine⁴], the derivatives are named by describing the substituted residue. For example, if cyclosporin A is modified, the derivative is named [γ-hydroxy-N-methyl-L-leucine⁴] cyclosporin A, and if cyclosporin B and cyclosporin C are modified, the derivatives are named [γ-hydroxy-N-methyl-L-leucine⁴] cyclosporin B and [γ-hydroxy-N-methyl-L-leucine⁴] cyclosporin C, respectively. Also, in the case that two or more residues are substituted, the derivatives are named in a similar manner. For example, if both of residues No. 3 and No. 4 are substituted, the derivative is named [N-methyl-D-alanine³] [γ-hydroxy-N-methyl-L-leucine⁴] cyclosporin A. If both of residues No. 4 and No. 9 are substituted, the derivative is named [γ-hydroxy-N-methyl-L-leucine⁴] [γ-hydroxy-N-methyl-L-leucine⁹] cyclosporin A. Also, if substitution at the residues No. 4 and No. 7 concurs, the derivative is described as [γ-hydroxy-N-methyl-L-leucine⁴] [alanine thiomide⁷, [⁷ψ⁸ CS—NH] cyclosporin A.

Regarding a peptolide in which the residue No. 8, D-alanine, is substituted with D-hydroxyisovaleric acid, forming an ester bond in its amino sequence, if the residue No. 4, N-methyl-L-leucine is converted to [γ-hydroxy-N-methyl-L-leucine⁴], the cyclosporin derivative is described as [L-threonine²] [L-leucine⁵] [γ-hydroxy-N-methyl-L-leucine⁴] [D-hydroxyisovaleric acid⁸] [L-leucine¹⁰] cyclosporin A. Further, as for a derivative of cyclosporin which is substituted with sulfur instead of a carbonyl oxygen at the residue No. 7, the name of the derivative may be cyclosporin 7-thioamide or [⁷ψ⁸ CS—NH] cyclosporin, according to different references (Helv. Chim. Acta. 74: 1953-1990, 1991; J. Org. Chem. 58: 673-677, 1993; J. Org. Chem. 59: 7249-7258, 1994).

Meanwhile, a common method for abbreviating amino acids is employed, that is, N-methyl-L-leucine is abbreviated by MeLeu, N-methyl-L-isoleucine by MeIle, N-methyl-L-Valine by MeVal, N-methyl-L-alanine by MeAla, N-methyl-L-norvaline by MeNva, L-leucine by Leu, L-isoleucine by Ile, sarcosine by Sar, L-serine by Ser, L-valine, Val, L-alanine by Ala, D-alanine by DAla, L-α-aminobutyric acid by Abu, L-threonine by Thr, and L-norvaline by Nva. In the invention, the term ‘cyclosporin derivatives’ generally refers to [γ-hydroxy-N-methyl-L-leucine⁴] cylosporin derivatives.

So far, possible development of cyclosporin as a hair-regrowth agent has been studied by many research groups. Particularly, research involving animal hair regrowth tests (Arch, Dermatol. Res., 1996, 288:408-410), human alopecia greata (J. Am. Acad. Dermatol., 1990, 22:242-250), human male pattern alopecia (J. Am. Acad. Dermatol., 1990, 22:251-253 and Skin Pharmacol., 1994, 7:101-104), and inhibition effect of hair loss by chemotherapy in animal models (Clin. Lab. Invest., 1995, 190:192-196 and Am. J. Pathol., 1997, 150:1433-1441) have been widely conducted. In comparative experiments on mouse's back, it is shown that cyclosporin has a hair regrowth effect about 100 times superior to minoxidil. Based on such findings, there have been attempts to utilize cyclosporin as a treatment for male pattern alopecia, and many applications for patents have been filed.

For example, Japanese Patent Publication Kokai Nos. Sho 60-243008, Sho 62-19512 and Sho 62-19513 disclose use of cyclosporin derivatives as a hair regrowth agent. Also, European Patent Publication No. 0414632 B1 discloses cyclosporin derivatives modified at residue No. 8, and PCT Patent Publication No. WO 93/17039 discloses isocyclosporin provided as a hair regrowth agent. In U.S. Pat. No. 5,807,820 and U.K. Patent No. 2,218,334 A, preparations containing cyclosporin with excellent transdermal absorption are suggested for new application of a hair regrowth agent. Furthermore, PCT Patent Publication No. WO 00/51558 discloses hair regrowth using immunosuppressive cyclosporin derivatives. These references do not include a structure of [γ-hydroxy-N-methyl-L-leucine⁴] cyclosporin A derivative claimed herein.

Although cyclosporin derivatives disclosed in the above references have good hair restoring effects versus common hair loss, they all show a strong immunosuppressive activity, limiting their application as hair regrowth agents. In addition to their severe side effect of immunosuppression, those cyclosporin derivatives disclosed in the prior art have a problem with their delivery to the skin and follicle.

### DISCLOSURE OF THE INVENTION

Therefore, the present invention has been made in view of the above problems, and it is an object of the present invention to provide compositions of carrier particles for use on the scalp and the skin, and methods of preparing them, by which penetration of a novel hair growth promoting agent to the follicle and the skin can be maximized, thereby exerting an excellent hair restoring effect. The present inventors found that as the hair growth promoting agent, [γ-hydroxy-N-methyl-L-leucine⁴] cyclosporin derivatives, which are modified at residue No. 4 in their amino acid sequence, uniquely exhibit an excellent hair restoring effect without immunosuppressive activity. Further, the inventors have studied to develop various formulations so that [γ-hydroxy-N-methyl-L-leucine⁴] cyclosporin derivatives may be well penetrated to the follicle and the skin. As a result, carriers having an excellent drug delivery effect, such as liposomes, microcapsules, microspheres, composite particles and emulsions, were manafactured. Those formulations can be used in manufacturing a hair restoring agent and an agent for the prevention of hair loss, although the cyclosporin derivatives are difficult agents to penetrate to the follicle and the skin, due to their high molecular weight.

More specifically, the invention is directed to compositions of carrier particles with a high degree of skin penetration and improved in vivo hair restoring effects. The compositions of carrier particles of the invention were prepared by encapsulating a non-immunosuppresive cyclosporin derivative, which is an active ingredient with hair restoration property, in carriers such as liposomes, microcapsules, microspheres, composite particles and emulsions. Since the compositions exhibit excellent penetration to the follicle and the skin, they are useful for hair restoring and for the prevention of hair loss.

In accordance with one aspect of the invention, the above and other objects can be accomplished by the provision of a composition of a carrier particle with a high degree of penetration to the follicle and the skin, and thus excellent hair restoring effects, and a method of preparing the same. The composition of the carrier particle of the invention is prepared by encapsulating one of [γ-hydroxy-N-methyl-L-leucine⁴] cyclosporin derivatives, represented by Chemical Formula 1 below, having an excellent hair restoring effect without immunosuppresive activity, in the carrier such as a liposome, microcapsule, microsphere, composite particle or emulsion. 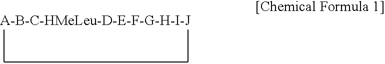
wherein:
A represents N-methyl-(4R)-4-[(E)-2-butenyl]-4-methyl-L-threonine, (2S,3R,4R,6E)-3-sulfhydryl-4-methyl-2-(methylamino)-6-octenoic acid or (2S,4R,6E)-3-oxo-4-methyl-2-(methylamino)-6-octenoic acid; B represents L-α-aminobutyric acid (Abu), L-alanine (Ala), L-threonine (Thr), L-valine (Val) or L-norvaline (Nva); C represents a D-amino acid represented by the general formula 1,
   CH₃NH—CH(R)—COOH [General formula 1]
in which R is one selected from the group consisting of hydrogen, C₁-C₆ straight or branched alkyl, alkenyl or alkynyl moieties, substituted or unsubstituted with one or more selected from the group consisting of amino, hydroxy, halo, haloalkyl, ester, alkoxy, cyano, nitro, alkylamino, and dialkylamino, and X—R′ represented by the general formula 2 below,
   —X—R′ [General formula 2]
in which X is oxygen or sulfur, and R′ is one selected from the group consisting of hydrogen, and C₁-C₆ straight or branched alkyl, alkenyl or alkynyl moieties, substituted or unsubstituted with one or more selected from the group consisting of amino, hydroxy, halo, haloalkyl, ester, alkoxy, cyano, nitro, alkylamino, and dialkylamino; HMeLeu represents γ-hydroxy N-methyl-L-leucine; D represents L-valine, L-norvaline or L-leucine; E represents N-methyl-L-leucine, y-hydroxy N-methyl-L-leucine, or L-leucine; F represents L-alanine or L-alanine thioamide ([⁷ψ⁸ CS—NH], NH—CHCH₃—CS—); G represents D-hydroxyisovaleric acid or a D-amino acid represented by the general formula 3,
   —NH—CH(CH₂R)—COOH [General formula 3]
in which R is hydrogen or X—R′ represented by the general formula 4,
   —X—R′ [General formula 4]
in which X is oxygen or sulfur, and R′ is one selected from the group consisting of hydrogen, and C₁-C₆ straight or branched alkyl, alkenyl or alkynyl moieties, substituted or unsubstituted with one or more selected from the group consisting of amino, hydroxy, halo, haloalkyl, ester, alkoxy, cyano, nitro, alkylamino, and dialkylamino; H represents N-methyl-L-leucine, y-hydroxy-N-methyl-L-leucine or L-leucine; I represents N-methyl-L-leucine, γ-hydroxy-N-methyl-L-leucine or L-leucine; and J represents N-methyl-L-valine or L-valine.

In accordance with another aspect of the invention, there is provided a composition of a carrier particle with a high degree of penetration to the follicle and the skin, and thus excellent hair restoring effects, and a method of preparing the same. The composition of the carrier particle of the invention is prepared by encapsulating one of [γ-hydroxy-N-methyl-L-leucine⁴] cyclosporin derivatives, represented by Chemical Formula 2 below, having an excellent hair restoring effect without immunosuppresive activity, in the carrier such as a liposome, microcapsule, microsphere, composite particle or emulsion. 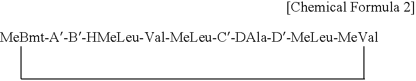
wherein:
MeBmt represents N-methyl-(4R)-4-[(E)-2-butenyl]-4-methyl-L-threonine; A′ represents L-α-aminobutyric acid, L-alanine, L-threonine, L-valine or L-norvaline; B′ represents N-methyl-D-alanine, D-2-(methylamino)pent-4-enoyl, N-methyl-D-aminobutyric acid, N-methyl-D-norvaline, D-2-(methylamino)hexa-4-ynoyl, D-2-(methylamino)pent-4-ynoyl, D-2-methylthio-sarcosine, N-methyl-O-propenyl-D-serine or N-methyl-D-serine; HMeLeu represents y-hydroxy-N-methyl-L-leucine; Val represents L-valine; MeLeu represents N-methyl-L-leucine; C′ represents L-alanine or L-alanine thioamide ([⁷ψ⁸ CS—NH], NH—CHCH₃—CS—); DAla represents D-alanine; D′ represents N-methyl-L-leucine, -hydroxy-N-methyl-L-leucine or L-leucine; and MeVal represents N-methyl-L-valine.

In accordance with another aspect of the invention, there is provided a composition of a carrier particle with a high degree of penetration to the follicle and the skin, and thus excellent hair restoring effects, and a method of preparing the same. The composition of the carrier particle of the invention is prepared by encapsulating one of [γ-hydroxy-N-methyl-L-leucine⁴] cyclosporin derivatives, represented by Chemical Formula 3 below, having an excellent hair restoring effect without immunosuppresive activity, in the carrier such as a liposome, microcapsule, microsphere, composite particle or emulsion. 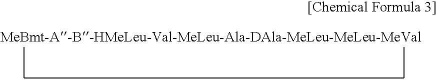
wherein:
MeBmt represents N-methyl-(4R)-4-[(E)-2-butenyl]-4-methyl-L-threonine; A″ represents L-α-aminobutyric acid, L-alanine, L-threonine, L-valine or L-norvaline; B″ represents N-methyl-D-alanine, D-2-(methylamino)pent-4-enoyl, N-methyl-D-aminobutyric acid, N-methyl-D-norvaline, D-2-(methylamino)hexa-4-ynoyl, D-2-(methylamino)pent-4-ynoyl, D-2-methylthio-sarcosine, N-methyl-O-propenyl-D-serine or N-methyl-D-serine; HMeLeu represents γ-hydroxy-N-methyl-L-leucine; Val represents L-valine; Ala represents L-alanine; MeLeu represents N-methyl-L-leucine; DAla represents D-alanine; and MeVal represents N-methyl-L-valine.

In accordance with another aspect of the invention, there is provided a composition of a carrier particle with a high degree of penetration to the follicle and the skin, and thus excellent hair restoring effects, and a method of preparing the same. The composition of the carrier particle of the invention is prepared by encapsulating [γ-hydroxy-N-methyl-L-leucine⁴] cylosporin A (Compound 1), having an excellent hair restoring effect without immunosuppresive activity, in the carrier such as a liposome, microcapsule, microsphere, composite particle or emulsion.

In accordance with another aspect of the invention, there is provided a composition of a carrier particle with a high degree of penetration to the follicle and the skin, and thus excellent hair restoring effects, and a method of preparing the same. The composition of the carrier particle of the invention is prepared by encapsulating [γ-hydroxy-N-methyl-L-leucine⁴] cylosporin C (Compound 2), having an excellent hair restoring effect without immunosuppresive activity, in the carrier such as a liposome, microcapsule, microsphere, composite particle or emulsion.

In accordance with another aspect of the invention, there is provided a composition of a carrier particle with a high degree of penetration to the follicle and the skin, and thus excellent hair restoring effects, and a method of preparing the same. The composition of the carrier particle of the invention is prepared by encapsulating [N-methyl-D-alanine³] [γ-hydroxy-N-methyl-L-leucine⁴] cyclosporin A (Compound 3), having an excellent hair restoring effect without immunosuppresive activity, in the carrier such as a liposome, microcapsule, microsphere, composite particle or emulsion.

In accordance with another aspect of the invention, there is provided a composition of a carrier particle with a high degree of penetration to the follicle and the skin, and thus excellent hair restoring effects, and a method of preparing the same. The composition of the carrier particle of the invention is prepared by encapsulating [γ-hydroxy-N-methyl-L-leucine4] [γ-hydroxy-N-methyl-L-leucine9] cyclosporin A (Compound 4), having an excellent hair restoring effect without immunosuppresive activity, in the carrier such as a liposome, microcapsule, microsphere, composite particle or emulsion.

In accordance with another aspect of the invention, there is provided a composition of a carrier particle with a high degree of penetration to the follicle and the skin, and thus excellent hair restoring effects, and a method of preparing the same. The composition of the carrier particle of the invention is prepared by encapsulating [γ-hydroxy-N-methyl-L-leucine⁴] [alanine thiomide⁷] cyclosporin A (or [⁷Ψ⁸ CS—NH] cyclosporin A) (Compound 5), having an excellent hair restoring effect without immunosuppresive activity, in the carrier such as a liposome, microcapsule, microsphere, composite particle or emulsion.

In accordance with another aspect of the invention, there is provided a composition of a carrier particle with a high degree of penetration to the follicle and the skin, and thus excellent hair restoring effects, and a method of preparing the same. The composition of the carrier particle of the invention is prepared by encapsulating [L-threonine]²[L-leucine]⁵ [γ-hydroxy-N-methyl-L-leucine⁴] [D-hydroxyisovaleric acid]⁸[L-leucine]¹⁰ cyclosporin A (Compound 6), having an excellent hair restoring effect without immunosuppresive activity, in the carrier such as a liposome, microcapsule, microsphere, composite particle or emulsion.

In accordance with yet another aspect of the invention, there is provided a composition of a carrier particle with a high degree of penetration to the follicle and the skin, and thus excellent hair restoring effects, and a method of preparing the same. The composition of the carrier particle of the invention is prepared by encapsulating [γ-hydroxy-N-methyl-L-leucine⁴] [D-serine⁸] cyclosporin A, having an excellent hair restoring effect without immunosuppresive activity, in the carrier such as a liposome, microcapsule, microsphere, composite particle or emulsion.

With regard to the composition of the carrier particle loading a [γ-hydroxy-N-methyl-L-leucine⁴] cyclosporin derivative, the non-immunosuppressive cyclosporin derivative, which has a high degree of penetration to the follicle and the skin, and thus excellent hair restoring effect, and the method of preparing the same, the present invention is more directed to a liposome composition and a method of preparing the same. Preferably, the liposome is prepared as follows. Amphiphilic molecules and a cyclosporin derivative are dissolved in organic solvent. The organic solvent is evaporated at ambient temperature, giving a mixture of dry lipid film consisting of the amphiphilic molecules and the cyclosporin derivative. Then, a certain amount of an aqueous solution is added to hydrate the dry phospholipid film. The resultant film is homogenized at 500 bar using a mechanical dispersion instrument.

With regard to the composition of the carrier particle loading a [γ-hydroxy-N-methyl-L-leucine⁴] cyclosporin derivative, the non-immunosuppressive cyclosporin derivative, which has a high degree of penetration to the follicle and the skin, and thus excellent hair restoring effect, and the method of preparing the same, the present invention is more directed to a microcapsule composition and a method of preparing the same. Preferably, the microcapsule is prepared as follows. A cyclosporin derivative is dissolved in an oil phase. The oil phase is emulsified in an aqueous solution. Then, the capsule wall materials in the aqueous phase of the emulsion are subjected to a chemical reaction.

With regard to the composition of the carrier particle loading a [γ-hydroxy-N-methyl-L-leucine⁴] cyclosporin derivative, the non-immunosuppressive cyclosporin derivative, which has a high degree of penetration to the follicle and the skin, and thus excellent hair restoring effect, and the method of preparing the same, the present invention is more directed to a microsphere composition and a method of preparing the same. Preferably, the microsphere is prepared as follows. A cyclosporin derivative and a polymer are dissolved in an oil phase. The oil phase is dispersed in a second immiscible phase. The oil phase is then evaporated.

With regard to the composition of the carrier particle loading a [γ-hydroxy-N-methyl-L-leucine⁴] cyclosporin derivative, the non-immunosuppressive cyclosporin derivative, which has a high degree of penetration to the follicle and the skin, and thus excellent hair restoring effect, and the method of preparing the same, the present invention is more directed to a composite particle composition and a method of preparing the same. Preferably, the composite particle is prepared as follows. A cyclosporin derivative and surfactant are mixed in an aqueous phase. The solution is forcibly dispersed using a mechanical dispersion instrument.

With regard to the composition of the carrier particle loading a [γ-hydroxy-N-methyl-L-leucine⁴] cyclosporin derivative, the non-immunosuppressive cyclosporin derivative, which has a high degree of penetration to the follicle and the skin, and thus excellent hair restoring effect, and the method of preparing the same, the present invention is more directed to an emulsion composition and a method of preparing the same. Preferably, the emulsion is prepared as follows. A cyclosporin derivative in an oil phase is emulsified in an aqueous phase containing an emulsifying agent.

With regard to the composition of the carrier particle loading a [γ-hydroxy-N-methyl-L-leucine⁴] cyclosporin derivative, the non-immunosuppressive cyclosporin derivative, which has a high degree of penetration to the follicle and the skin, and thus excellent hair restoring effect, and the method of preparing the same, the present invention is more directed to compositions for use on hair and methods of preparing the same. The liposomes, microcapsules, microspheres, composite particles or emulsions carrying the cyclosporin derivatives may be applied in preparing the compostions for use on hair.

In the invention, [γ-hydroxy-N-methyl-L-leucine⁴] cyclosporin derivatives, the non-immunosuppressive hair-restoring agent, are encapsulated in carrier particles such as liposomes, microcapsules, microspheres, composite particles and emulsions, thereby improving penetration to the follicle and the skin. As revealed in hair growth tests, these encapsulated cyclosporin derivatives showed an excellent hair restoring effect.

Liposomes of the invention can be prepared by dispersing a cyclosporin derivative and amphiphilic molecules such as phospholipids or other bilayer-forming molecules in an aqueous phase. As for phospholipids, phosphatidylcholine (PC), phosphatidylethanolamine (PE), phosphatidylserine (PS), phosphatidylglycerol (PG), phosphatidic acid (PA), phosphatidylinositol (PI), and derivatives thereof may be used. The other amphiphilic molecules which form bilayers include surfactants such as acyl Nₙ,Nₙ-dimethyl-1,n-diamino alkyl (n=2-10, 8-30 carbon atoms in the alkyl group), dialkyl dimethyl ammonium salt (12-18 carbon atoms in the alkyl chain, anion is Cl⁻ or Br⁻) and monoalkyl trimethyl ammonium salt (12-22 carbon atoms in the alkyl chain, anion is Cl⁻ or Br⁻), and fatty acids (8-30 carbon atoms in the alkyl chain). As for instruments for dispersion, a colloid mill depending on the friction force of a grinding stone which is rotated at high speed, a roll mill depending on the shear forces generated between adjacent rolls, in which several metal rolls are successively rotated, and a sonicator depending on mechanical disruption by ultrasonic energy may be used. Also, a high-pressure dispersion instrument (microfluidizer, Microfluidics Corp., USA) can be used. With this instrument, dispersion is achieved by shear stress, collision energy and cavitation, generated by flowing a liquid through a narrow orifice under high pressure. Other available instruments for homogenization may include Ultra Turrax (Janke and Kunkel, Germany), Nanoget (Nanoget Engineering, Germany) and Brogli (Italy). For preparing liposomes, a weight ratio of a cyclosporin derivative to amphiphilic molecules is 1/100 to 1/1, preferably 1/10 to 7/10, most preferably 1/5 to 1/2. If the cyclosporin derivative is employed at a weight ratio lower than these ranges, its hair restoring effect cannot be obtained. On the other hand, if the ratio is higher, liposomes fail to be formed. The weight % of amphiphilic molecules in a liposome suspension is 1 to 40%, preferably 2 to 30%, most preferably 3 to 20%. If the amphiphilic molecules are employed at a weight % lower than these ranges, it is hard to apply the cyclosporin derivative at the amount which exhibits its hair restoring effect into a composition for use on human hair. On the other hand, if the content is higher, a liposome is not likely to be formed since a dispersed state is poor upon liposome preparation.

Microcapsules of the invention can be prepared as follows. In the microcapsules are encapsulated [γ-hydroxy-N-methyl-L-leucine⁴] cyclosporin derivatives which are non-immunosuppressive hair-restoring agents. A cyclosporin derivative in an oil phase is emulsified in an aqueous phase in which wall materials of a microcapsule are dissolved. The wall materials in the aqueous phase are subjected to cross-linking or a condensation reaction, preparing the microcapsule. As for the wall materials, gelatin, carboxymethyl-cellulose (CMC), alginate, Arabic gum, acacia gum, methylol melamine, methylol urea, and derivatives thereof may be used. A cross-linking agent may include formaldehyde and glutaraldehyde. For oil phase, silicone oil such as polydimethyl siloxane (viscosity: 1.5×10⁻⁶ m²/s-2.5×10⁻¹ m²/s; Trade name: DC Fluid 200 (Dow Corning Co.), Abil 10, Abil K03 (Goldschmidt), Rhodorsil 47 V 10, Silbione 70047 V 10, Silbione 70047 V 100, Silbione 70047 V 300 (Rhone Poulenc), and Siliconee Oil L45 (Union Carbide)), plant or animal oil such as sweet almond oil, avocado oil, castor oil, olive oil, jojoba oil, sunflower oil, wheat germ oil, sesame oil, ground nut oil, raisin seed oil, sova oil, rape seed oil, safflower oil, coconut oil, corn oil, hazelnut oil, palm oil and apricot-kernel oil, mineral oil such as fluid paraffin, synthetic oil such as caprylic/capric triglycerides and triglycerides (C₁₀-C₁₈), and fatty acid triglyceride may be used.

Microspheres of the invention can be prepared as follows. In the microspheres are encapsulated [γ-hydroxy-N-methyl-L-leucine⁴] cyclosporin derivatives which are non-immunosuppressive hair-restoring agents. An oil phase in which a polymer is dissolved is emulsified in an aqueous phase in which an emulsifying agent is dissolved, thereby giving an O/W emulsion. A spontaneous reciprocal diffusion between the oil phase and the aqueous phase occurs, making the polymer insoluble. As a result, a polymer microsphere is formed, and the organic solvent is evaporated. As for the polymer, poly(lactic acid) (PLA), poly(glycolic acid) (PGA), poly(lactic acid-co-glycolic acid) (PLGA), poly(E-caprolactone) (PECL), and cellulose-acetate phthalate may be used. For the oil phase, dichloromethane, chloroform, acetone, a mixture of dichloromethane/acetone, and a mixture of acetone/propylene glycol dicaprylate dicaprate may be used. The emulsifying agent includes polyvinyl alcohol and gelatin.

Composite particles of the invention, that is, [γ-hydroxy-N-methyl-L-leucine⁴] cyclosporin derivative/surfactant composite particles can be prepared as follows. The cyclosporin derivative, a surfactant and distilled water are measured out at respective desired amounts, and they are mixed. The solution was homogenized using a mechanical dispersion instrument, thereby preparing a fine composite particle. The surfactant serves as a stabilizer for dispersion of the cyclosporin derivative.

As for the surfactant, anionic, cationic, nonionic or amphiphilic surfactants can be used. The anionic surfactant includes alkyl sulphates, alkyl ether sulphates, alkaryl sulphonates, alkanoyl isethionates, alkyl succinates, alkyl sulphosuccinates, N-alkoylsarcosinates, alkyl phosphates, alkyl ether phosphates, alkyl ether carboxylates, alpha-olefin sulphonates, especially sodium, magnesium, and ammonium salts thereof, and mono-, di-, and tri-ethanolamine salts thereof. In these surfactants, alkyl and acyl groups have generally 8 to 18 carbon atoms, and are unsaturated. Alkyl ether sulphates, alkyl ether phosphates, and alkyl ether carboxylates have respectively 1 to 10 ethylene oxide or propylene oxide groups. Particular examples of the anionic surfactants include sodium oleyl succinate, ammonium lauryl sulphosuccinate, ammonium lauryl sulphate, sodium dodecylbenzene sulphonate, triethanolamine dodecylbenzene sulphonate, sodium cocoyl isethionate, sodium lauroyl isethionate, sodium N-lauryl sarcosinate. The most preferred anionic surfactants are sodium lauryl sulphate, triethanolamine lauryl sulphate, triethanolamine monolauryl phosphate, sodium lauryl ether sulphate (having 1 to 3 ethylene oxide groups), ammonium lauryl sulphate, sodium lauryl ether sulphate (having 1 to 3 ethylene oxide groups).

As for a cationic surfactant, a quaternary ammonium salt represented by the following structure can be used.

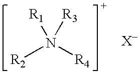

In the above formula, among R₁, R₂, R₃ and R₄, one or two is/are linear alkyl or branched alkyl or hydroxy alkyl groups (C₈-C₂₂), and the others are alkyl groups having 1 to 3 carbon atoms, hydroxy alkyl groups, benzyl groups or polyoxyethylene groups. X is a halogen atom or an alkyl sulfate group having one or two carbon atoms. Among the quaternary ammonium salts, distearyldimethylammonium chloride, stearyl trimethylammonium methosulfate, stearyltrimethylammonium chloride, stearyldimethylbenzylammonium chloride, docosyltrimethylammonium methosulfate, docosyltrimethylammonium chloride, docosyldimethylbenzylammonium chloride, didocosyldimethylammonium chloride, lauryldiethylbenzylammonium chloride, lauryltrimethylammonium bromide, distearylmethylhydroxymethyl chloride, cetyltrimethylammonium chloride, N-stearyl-N,N,N-tri(polyoxyethylene)ammonium chloride, cetyltriethylammonium bromide or stearyldimethylammonium chloride is preferably used.

As for a nonionic surfactant, a primary or secondary fatty alcohol (C₈-C₁₈, linear or branched chain) or a product obtained from a condensation reaction between phenol and alkylene oxide (mainly having 6 to 30 ethylene oxide groups) can be used. Other nonionic surfactants suitable for use in the invention include alkyl-polyglycosides, mono-, di-alkyl alkanol amides, and coco mono-isopropanolamide.

Suitable amphiphilic surfactants used in the invention may include alkyl amine oxides, alkyl betaines, alkyl amidoprophyl betaines, alkyl sulphobetaines, alkyl glycinates, alkyl carboxyglycinates, alkyl amphopropionates, alkyl amphoglycinates, and alkyl amidopropyl hydroxylsultaines. In these surfactants, the alkyl and acyl groups have 8 to 19 carbon atoms. For example, lauryl amine oxide or cocodimethyl sulphopropyl betaine can be used. Lauryl betaine, cocamidopropyl betaine or sodium cocamphopripionate is preferably used.

For preparing the composite particles of the invention, in a mixture of [γ-hydroxy-N-methyl-L-leucine⁴] cyclosporin derivative/surfactant/water, a weight % of the cyclosporin derivative is 0.01 to 35%, preferably 1 to 30%, most preferably 2 to 25%. A weight ratio of the surfactant to the cyclosporin derivative is 1/100 to 100/1, preferably 1/50 to 50/1, most preferably 1/25 to 25/1. If the contents of cyclosporin derivative and surfactant employed are lower than these ranges, it cannot be ensured that the final cyclosporin derivative content is in a high range of 0.1 to 3% which exhibits hair restoring effect, since the cyclosporin derivative/surfactant composite particles are diluted upon preparing a composition for use on human hair. On the other hand, if the contents are higher, it is hard to disperse them using a mechanical dispersion instrument.

As for the dispersion instrument, a colloid mill depending on the friction force of a grinding stone which is rotated at high speed, a roll mill depending on the shear forces generated between adjacent rolls, in which several metal rolls are successively rotated, or a sonicator depending on mechanical disruption by ultrasonic energy may be used. Also, a high-pressure dispersion instrument (microfluidizer, Microfluidics Corp., USA) can be used. With the instrument, dispersion is achieved by shear stress, collision energy and cavitation, generated by flowing a liquid through a narrow orifice under high pressure. Other available instruments for homogenization may include Ultra Turrax (Janke and Kunkel, Germany), Nanoget (Nanoget Engineering, Germany) and Brogli (Italy) and mechanical dispersion instruments equivalent thereto.

Emulsions according to the invention contain cyclosporin derivatives, and the method of preparing them is as follows. An oil phase in which the cyclosporin derivative is dissolved is emulsified in an aqueous phase containing an emulsifying agent. As for the oil phase, plant or animal oil such as sweet almond oil, avocado oil, castor oil, olive oil, jojoba oil, sunflower oil, wheat germ oil, sesame oil, ground nut oil, raisin seed oil, sova oil, rape seed oil, safflower oil, coconut oil, corn oil, hazelnut oil, palm oil and apricot-kernel oil, mineral oil such as fluid paraffin, synthetic oil such as caprylic/capric triglycerides and triglycerides (C₁₀-C₁₈), and fatty acid triglyceride may be used.

For preparing the emulsions of the invention, a volume ratio of the oil phase to the aqueous phase is 0.01/1 to 1.2/1, preferably 0.05/1 to 1/1, most preferably 0.1/1 to 0.7/1. If the volume ratio of the oil phase to the aqueous phase is lower than these ranges, it cannot be ensured that the final cyclosporin derivative content is in a high range of 0.1 to 3% which exhibits hair restoring effect, since the emulsions containing cyclosporin derivatives are diluted upon preparing a composition for use on human hair. On the other hand, if the volume ratio is higher, it is hard to manufacture stable emulsions.

As for the emulsifying agent, substances which are relatively hydrophilic and have a surface activating ability, including polyvinyl alcohol, gelatin, polysorbate 80, sodium alginate, sodium oleate, methyl cellulose, albumin, sodium dodecyl sulfate, sodium lauryl sulfate, polysorbate 20 and fluroric (F68), and the cationic, anionic, amphiphilic, nonionic surfactants which are used in preparing the composite particles of the invention can be used. As for the dispersion instrument, a colloid mill, a roll mill, a sonicator, a high-pressure dispersion instrument (microfluidizer, Microfluidics Corp., USA), Ultra Turrax (Janke and Kunkel, Germany), Nanoget (Nanoget Engineering, Germany) and Brogli (Italy) and mechanical dispersion instruments equivalent thereto can be used.

In accordance with the invention, the carrier particles which are fine particles of several microns in size, such as liposomes, microcapsules, microspheres, composite particles and emulsions, in which a cyclosporin derivative according to the invention is encapsulated, advantageously show higher skin penetration and better in vivo hair restoring effect than those of free cyclosporin derivatives dissolved in organic solvent at a molecular level. Further, the carrier particles of the invention show good dispersion and phase stability over time in compositions for use on hair. Accordingly, it is advantageous that the carrier particles can prevent free forms of cyclosporin derivatives being poorly dispersed and phase-unstable over time, upon application to the hair cleaning compositions.

The fine carrier particles loading the cyclosporin derivatives manufactured in the invention have the following advantages over conventional solutions containing cyclosporin derivatives and cyclosporin powder which is not formulated.

1. High Degree of Penetration and Excellent Hair Restoring Effect

The carrier particles loading the cyclosporin derivatives, manufactured according to the methods of the invention derivatives, which are fine particles of several microns in size, exhibit a high degree of penetration to the skin through the follicle and excellent in vivo hair restoring effects, compared to conventional formulations.

2. Good Dispersion and Phase Stability Over Time

Since cyclosporin derivative powder in particle form, not formulated, is strongly hydrophobic, its dispersion in cleaning compositions is poor. In addition, the powder particles are likely to agglomerate even after forcible mechanical dispersion, thereby the powder particles are precipitated with time. That is, its phase stability is poor. On the other hand, the carrier particles loading cyclosporin derivatives of the invention are hydrophilic due to their surface characteristics. Accordingly, the dispersion state in compositions for use on human skin is good, and their phase stability is excellent.

In summary, the carrier particles loading the cyclosporin derivatives, manufactured according to the methods of the invention derivatives, exhibit excellence in terms of skin penetration, in vivo hair restoring effect, dispersion in compositions for use on human skin, and phase stability with time, compared to conventional solutions containing cyclosporin derivatives and cyclosporin powder which is not formulated.

Meanwhile, the carrier particles loading the cyclosporin derivatives, manufactured according to the methods of the invention derivatives can be applied in manufacturing compositions for use on hair, such as shampoos and rinses.

### BEST MODE FOR CARRYING OUT THE INVENTION

Preparation of Cyclosporin Derivatives

To prepare [γ-hydroxy-N-methyl-L-leucine⁴] cyclosporin derivatives, which have an excellent hair growth stimulating effect and exhibit no immunosuppressive activity, a bacterial strain, Sebekia benihana KCTC 9173 was employed. Cyclosporin derivatives having N-methyl-L-leucine at their amino acid No. 4 were dissolved in methanol. Each of thus prepared cyclosporin solutions was added at an amount of 100 mg/L to the culture medium 24 hrs after starting the main culture. The bacteria were further cultured for 72 hrs.

After 72 hours, the total culture solution was extracted with an equal volume of ethyl acetate, thereby collecting the sample. The organic solvent layer was concentrated. From the concentrated sample, [γ-hydroxy-N-methyl-L-leucine⁴] cyclosporin deriatives, which are a hair-restoring component with no immunosuppressive activity, were isolated and collected by means of high performance liquid chromatography, to serve as test samples for the following experiments. The isolation condition was as follows. A C-18 column was used. For the elution, a 100% solvent A was flowed for 2 min. The concentration of the solvent was lowered to 60%, and the 60% solvent was flowed over 4 min, and then the concentration was slowly lowered to 39%, over 60 min. The concentration of the solvent was then returned to 100%, and flowed for a further 5 min. The solvent A was 25% aqueous methanol. As the diluent solvent B, 100% acetonitrile was used.

Hereinafter, the present invention will be described in detail, in conjunction with various examples. Those skilled in the art will realize that these examples are provided only for illustrative purposes, and the present invention is not to be construed as being limited to those examples.

Formulations

Formulation 1:

Preparation of a Liposome Carrying a Cyclosporin Derivative

A method of encapsulating a cyclosporin derivative into a liposome of the invention is as follows. Amphiphilic molecules composing the liposome used herein include phospholipids such as phosphatidylcholine (PC), phosphatidylethanolamine (PE) and phosphatidylinositol (PI), cholesterol, cationic surfactants such as stearamidopropyldimethylamine (SAPDA), distearyldimethyl ammoniumchloride (DSDAC) and stearyltrimethylammoniumchloride (STAC), and stearic acid (SA). The amphiphilic molecules and cyclosporin derivative were dissolved in an organic solvent such as acetone, methanol, ethanol, isopropanol, or chloroform. The organic solvent was evaporated at ambient temperature, giving a mixture of dry lipid film consisting of amphiphilic molecules and the cyclosporin derivative. Then, a certain amount of an aqueous solution was added to hydrate the dry phospholipid film. The resultant film was homogenized at 500 bar using a microfluidizer.

In the following, the invention is described in more detail.

### EXAMPLES 1 TO 7

### Preparation of a Liposome Carrying [γ-hydroxy-N-methyl-L-leucine⁴] Cyclosporin A, Compound 1

According to the method described in Formulation 1, liposome suspensions carrying the cyclosporin derivative were prepared, with varying compositions of the ingredients shown in Table 1.

TABLE 1
Formulation of liposome

| | (unit: weight %) | | | | | | |
|---|---|---|---|---|---|---|---|
| Ingredients | Ex. 1 | Ex. 2 | Ex. 3 | Ex. 4 | Ex. 5 | Ex. 6 | Ex. 7 |
| PC | 10 | 8 | 8 | 8 | — | — | — |
| PE | — | 2 | — | — | — | — | — |
| PI | — | — | 2 | — | — | — | — |
| Cholesterol | — | — | — | 2 | — | — | — |
| DSDMAC | — | — | — | — | 10 | — | — |
| STAC | — | — | — | — | — | 5 | — |
| SAPDA | — | — | — | — | — | — | 5 |
| SA | — | — | — | — | — | 5 | 5 |
| [γ-hydroxy- | 5 | 5 | 5 | 5 | 5 | 5 | 5 |
| N-methyl-L- | | | | | | | |
| leucine⁴] | | | | | | | |
| cyclosporin A | | | | | | | |
| Water | 85 | 85 | 85 | 85 | 85 | 85 | 85 |

### EXAMPLES 8 TO 14

### Preparation of a Liposome Carrying [γ-hydroxy-N-methyl-L-leucine⁴] Cyclosporin C, Compound 2

According to the method described in Formulation 1, liposome suspensions carrying the cyclosporin derivative were prepared, with varying compositions of the ingredients shown in Table 2.

TABLE 2
Formulation of liposome

| | (unit: weight %) | | | | | | |
|---|---|---|---|---|---|---|---|
| | Ex. | Ex. | Ex. | Ex. | Ex. | Ex. | Ex. |
| Ingredients | 8 | 9 | 10 | 11 | 12 | 13 | 14 |
| PC | 10 | 8 | 8 | 8 | — | — | — |
| PE | — | 2 | — | — | — | — | — |
| PI | — | — | 2 | — | — | — | — |
| Cholesterol | — | — | — | 2 | — | — | — |
| DSDMAC | — | — | — | — | 10 | — | — |
| STAC | — | — | — | — | — | 5 | — |
| SAPDA | — | — | — | — | — | — | 5 |
| SA | — | — | — | — | — | 5 | 5 |
| [γ-hydroxy- | 5 | 5 | 5 | 5 | 5 | 5 | 5 |
| N-methyl-L- | | | | | | | |
| leucine⁴] | | | | | | | |
| cyclosporin C | | | | | | | |
| Water | 85 | 85 | 85 | 85 | 85 | 85 | 85 |

### EXAMPLES 15 TO 21

### Preparation of a Liposome Carrying [N-methyl-D-alanine³][γ-hydroxy-N-methyl-L-leucine⁴] Cyclosporin A, Compound 3

According to the method described in Formulation 1, liposome suspensions carrying the cyclosporin derivative were prepared, with varying compositions of the ingredients shown in Table 3.

TABLE 3
Formulation of liposome

| | (unit: weight %) | | | | | | |
|---|---|---|---|---|---|---|---|
| Ingredients | Ex. 15 | Ex. 16 | Ex. 17 | Ex. 18 | Ex. 19 | Ex. 20 | Ex. 21 |
| PC | 10 | 8 | 8 | 8 | — | — | — |
| PE | — | 2 | — | — | — | — | — |
| PI | — | — | 2 | — | — | — | — |
| Cholesterol | — | — | — | 2 | — | — | — |
| DSDMAC | — | — | — | — | 10 | — | — |
| STAC | — | — | — | — | — | 5 | — |
| SAPDA | — | — | — | — | — | — | 5 |
| SA | — | — | — | — | — | 5 | 5 |
| [N-methyl-D-alanine³][γ- | 5 | 5 | 5 | 5 | 5 | 5 | 5 |
| hydroxy-N-methyl-L-leucine⁴] | | | | | | | |
| cyclosporin A | | | | | | | |
| Water | 85 | 85 | 85 | 85 | 85 | 85 | 85 |

### EXAMPLES 22 TO 28

### Preparation of a Liposome Carrying [γ-hydroxy-N-methyl-L-leucine⁴] [γ-hydroxy-N-methyl-L-leucine⁹] Cyclosporin A, Compound 4

According to the method described in Formulation 1, liposome suspensions carrying the cyclosporin derivative were prepared, with varying compositions of the ingredients shown in Table 4.

TABLE 4
Formulation of liposome

| | (unit: weight %) | | | | | | |
|---|---|---|---|---|---|---|---|
| Ingredients | Ex. 22 | Ex. 23 | Ex. 24 | Ex. 25 | Ex. 26 | Ex. 27 | Ex. 28 |
| PC | 10 | 8 | 8 | 8 | — | — | — |
| PE | — | 2 | — | — | — | — | — |
| PI | — | — | 2 | — | — | — | — |
| Cholesterol | — | — | — | 2 | — | — | — |
| DSDMAC | — | — | — | — | 10 | — | — |
| STAC | — | — | — | — | — | 5 | — |
| SAPDA | — | — | — | — | — | — | 5 |
| SA | — | — | — | — | — | 5 | 5 |
| [γ-hydroxy-N-methyl-L- | 5 | 5 | 5 | 5 | 5 | 5 | 5 |
| leucine⁴] [γ-hydroxy-N- | | | | | | | |
| methyl-L-leucine⁹] | | | | | | | |
| cyclosporin A | | | | | | | |
| Water | 85 | 85 | 85 | 85 | 85 | 85 | 85 |

### EXAMPLES 29 TO 35

### Preparation of a Liposome Carrying [γ-hydroxy-N-methyl-L-leucine⁴] [alanine thiomide⁷] Cyclosporin A (or [⁷Ψ⁸ CS—NH] Cyclosporin A), Compound 5

According to the method described in Formulation 1, liposome suspensions carrying the cyclosporin derivative were prepared, with varying compositions of the ingredients shown in Table 5.

TABLE 5
Formulation of liposome

| | (unit: weight %) | | | | | | |
|---|---|---|---|---|---|---|---|
| Ingredients | Ex. 29 | Ex. 30 | Ex. 31 | Ex. 32 | Ex. 33 | Ex. 34 | Ex. 35 |
| PC | 10 | 8 | 8 | 8 | — | — | — |
| PE | — | 2 | — | — | — | — | — |
| PI | — | — | 2 | — | — | — | — |
| Cholesterol | — | — | — | 2 | — | — | — |
| DSDMAC | — | — | — | — | 10 | — | — |
| STAC | — | — | — | — | — | 5 | — |
| SAPDA | — | — | — | — | — | — | 5 |
| SA | — | — | — | — | — | 5 | 5 |
| [γ-hydroxy-N-methyl-L- | 5 | 5 | 5 | 5 | 5 | 5 | 5 |
| leucine⁴] [alanine thiomide⁷] | | | | | | | |
| cyclosporin A | | | | | | | |
| Water | 85 | 85 | 85 | 85 | 85 | 85 | 85 |

### EXAMPLES 36 TO 42

### Preparation of a Liposome Carrying [L-threonine]²[L-leucine]⁵[-hydroxy-N-methyl-L-leucine⁴] [D-hydroxyisovaleric Acid]⁸[L-leucine]¹⁰ Cyclosporin A, Compound 6

According to the method described in Formulation 1, liposome suspensions carrying the cyclosporin derivative were prepared, with varying compositions of the ingredients shown in Table 6.

TABLE 6
Formulation of liposome

| | (unit: weight %) | | | | | | |
|---|---|---|---|---|---|---|---|
| Ingredients | Ex. 36 | Ex. 37 | Ex. 38 | Ex. 39 | Ex. 40 | Ex. 41 | Ex. 42 |
| PC | 10 | 8 | 8 | 8 | — | — | — |
| PE | — | 2 | — | — | — | — | — |
| PI | — | — | 2 | — | — | — | — |
| Cholesterol | — | — | — | 2 | — | — | — |
| DSDMAC | — | — | — | — | 10 | — | — |
| STAC | — | — | — | — | — | 5 | — |
| SAPDA | — | — | — | — | — | — | 5 |
| SA | — | — | — | — | — | 5 | 5 |
| [L-threonine]²[L-leucine]⁵[γ- | 5 | 5 | 5 | 5 | 5 | 5 | 5 |
| hydroxy-N-methyl-L-leucine⁴] | | | | | | | |
| [D-hydroxyisovaleric acid]⁸[L- | | | | | | | |
| leucine]¹⁰ cyclosporin A | | | | | | | |
| Water | 85 | 85 | 85 | 85 | 85 | 85 | 85 |

### EXAMPLES 43 TO 49

### Preparation of a Liposome Carrying [γ-hydroxy-N-methyl-L-leucine⁴] Cyclosporin A, Compound 1

According to the method described in Formulation 1, liposome suspensions carrying the cyclosporin derivative were prepared, with varying compositions of the ingredients shown in Table 7.

TABLE 7
Formulation of liposome

| | (unit: weight %) | | | | | | |
|---|---|---|---|---|---|---|---|
| Ingredients | Ex. 43 | Ex. 44 | Ex. 45 | Ex. 46 | Ex. 47 | Ex. 48 | Ex. 49 |
| PC | 10 | 8 | 8 | 8 | — | — | — |
| PE | — | 2 | — | — | — | — | — |
| PI | — | — | 2 | — | — | — | — |
| Cholesterol | — | — | — | 2 | — | — | — |
| DSDMAC | — | — | — | — | 10 | — | — |
| STAC | — | — | — | — | — | 5 | — |
| SAPDA | — | — | — | — | — | — | 5 |
| SA | — | — | — | — | — | 5 | 5 |
| [γ-hydroxy-N-methyl-L- | 0.1 | 0.1 | 0.1 | 0.1 | 0.1 | 0.1 | 0.1 |
| leucine⁴] cyclosporin A | | | | | | | |
| Water | 89.9 | 89.9 | 89.9 | 89.9 | 89.9 | 89.9 | 89.9 |

### EXAMPLES 50 TO 56

### Preparation of a Liposome Carrying [γ-hydroxy-N-methyl-L-leucine⁴] Cyclosporin C, Compound 2

According to the method described in Formulation 1, liposome suspensions carrying the cyclosporin derivative were prepared, with varying compositions of the ingredients shown in Table 8.

TABLE 8
Formulation of liposome

| | (unit: weight %) | | | | | | |
|---|---|---|---|---|---|---|---|
| Ingredients | Ex. 50 | Ex. 51 | Ex. 52 | Ex. 53 | Ex. 54 | Ex. 55 | Ex. 56 |
| PC | 10 | 8 | 8 | 8 | — | — | — |
| PE | — | 2 | — | — | — | — | — |
| PI | — | — | 2 | — | — | — | — |
| Cholesterol | — | — | — | 2 | — | — | — |
| DSDMAC | — | — | — | — | 10 | — | — |
| STAC | — | — | — | — | — | 5 | — |
| SAPDA | — | — | — | — | — | — | 5 |
| SA | — | — | — | — | — | 5 | 5 |
| [γ-hydroxy-N-methyl-L- | 0.1 | 0.1 | 0.1 | 0.1 | 0.1 | 0.1 | 0.1 |
| leucine⁴] cyclosporin C | | | | | | | |
| Water | 89.9 | 89.9 | 89.9 | 89.9 | 89.9 | 89.9 | 89.9 |

### EXAMPLES 57 TO 63

### Preparation of a Liposome Carrying [N-methyl-D-alanine³][γ-hydroxy-N-methyl-L-leucine⁴] Cyclosporin A, Compound 3

According to the method described in Formulation 1, liposome suspensions carrying the cyclosporin derivative were prepared, with varying compositions of the ingredients shown in Table 9.

TABLE 9
Formulation of liposome

| | (unit: weight %) | | | | | | |
|---|---|---|---|---|---|---|---|
| Ingredients | Ex. 57 | Ex. 58 | Ex. 59 | Ex. 60 | Ex. 61 | Ex. 62 | Ex. 63 |
| PC | 10 | 8 | 8 | 8 | — | — | — |
| PE | — | 2 | — | — | — | — | — |
| PI | — | — | 2 | — | — | — | — |
| Cholesterol | — | — | — | 2 | — | — | — |
| DSDMAC | — | — | — | — | 10 | — | — |
| STAC | — | — | — | — | — | 5 | — |
| SAPDA | — | — | — | — | — | — | 5 |
| SA | — | — | — | — | — | 5 | 5 |
| [N-methyl-D-alanine³][γ- | 0.1 | 0.1 | 0.1 | 0.1 | 0.1 | 0.1 | 0.1 |
| hydroxy-N-methyl-L-leucine⁴] | | | | | | | |
| cyclosporin A | | | | | | | |
| Water | 89.9 | 89.9 | 89.9 | 89.9 | 89.9 | 89.9 | 89.9 |

### COMPARATIVE EXAMPLE 1

Preparation of a Solution of a Cyclosporin Derivative

A cyclosporin derivative was dissolved in ethanol and acetone, preparing a 5% cyclosporin solution. In testing a hair growth promoting ability, a 0.1% cyclosporin solution was employed, and the solution was prepared by dissolving a cyclosporin derivative in a 50% aqueous ethanol solution.

Formulation 2:

Preparation of a Microcapsule Carrying a Cyclosporin Derivative

A method of encapsulating a cyclosporin derivative into a microcapsule of the invention is as follows. A cyclosporin derivative was dissolved in olive oil and medium chain triglyceride (MCT, Takasago Int'l Corp.). The solution was added to a 3% aqueous gelatin solution (Sigma, USA), and emulsified at 50° C. at 2000 rpm for 10 min. Then, the solution was added with a 3% aqueous carboxylmethyl cellulose (CMC, medium viscosity) (Sigma, USA) solution or a 3% aqueous Arabic gum (MW 250,000) (Sigma, USA) solution, and additionally emulsified for 10 min. The resulting solution was added with a 10% aqueous acetic acid solution, and adjusted to pH 4.4. The emulsified solution was cooled to a temperature of approximately 10C and stirred for 30 min. The solution was then added with a 25% aqueous glutaraldehyde (GA) solution and stirred at 200 rpm for 4 hrs at ambient temperature, curing the capsule.

### EXAMPLES 64 TO 69

### Preparation of a Microcapsule Carrying [γ-hydroxy-N-methyl-L-leucine⁴] Cyclosporin A, Compound 1

According to the method described in Formulation 2, microcapsule suspensions carrying the cyclosporin derivative were prepared, with varying compositions of the ingredients shown in Table 10.

TABLE 10
Formulation of microcapsule

| | (unit: weight %) | | | | | |
|---|---|---|---|---|---|---|
| Ingredients | Ex. 64 | Ex. 65 | Ex. 66 | Ex. 67 | Ex. 68 | Ex. 69 |
| Olive oil | 20 | — | 20 | — | 20 | — |
| MCT | — | 20 | — | 20 | — | 20 |
| [γ-hydroxy-N-methyl-L-leucine⁴] | 5 | 5 | 5 | 5 | 5 | 5 |
| cyclosporin A | | | | | | |
| Gelatin A (75-100 Bloom) | 30 | 30 | — | — | — | — |
| Gelatin A (300 Bloom) | — | — | 30 | 30 | 30 | 30 |
| CMC | 30 | 30 | 30 | 30 | — | — |
| Arabic gum | — | — | — | — | 30 | 30 |
| Acetic acid (10 w % aqueous solution) | 1 | 1 | 1 | 1 | 1 | 1 |
| GA (25 w % aqueous solution) | 1.5 | 1.5 | 1.5 | 1.5 | 1.5 | 1.5 |
| Water | 12.5 | 12.5 | 12.5 | 12.5 | 12.5 | 12.5 |

### EXAMPLES 70 TO 75

### Preparation of a Microcapsule Carrying [γ-hydroxy-N-methyl-L-leucine⁴] Cyclosporin C, Compound 2

According to the method described in Formulation 2, microcapsule suspensions carrying the cyclosporin derivative were prepared, with varying compositions of the ingredients shown in Table 11.

TABLE 11
Formulation of microcapsule

| | (unit: weight %) | | | | | |
|---|---|---|---|---|---|---|
| Ingredients | Ex. 70 | Ex. 71 | Ex. 72 | Ex. 73 | Ex. 74 | Ex. 75 |
| Olive oil | 20 | — | 20 | — | 20 | — |
| MCT | — | 20 | — | 20 | — | 20 |
| [γ-hydroxy-N-methyl-L-leucine⁴] | 5 | 5 | 5 | 5 | 5 | 5 |
| cyclosporin C | | | | | | |
| Gelatin A (75-100 Bloom) | 30 | 30 | — | — | — | — |
| Gelatin A (300 Bloom) | — | — | 30 | 30 | 30 | 30 |
| CMC | 30 | 30 | 30 | 30 | — | — |
| Arabic gum | — | — | — | — | 30 | 30 |
| Acetic acid (10 w % aqueous solution) | 1 | 1 | 1 | 1 | 1 | 1 |
| GA (25 w % aqueous solution) | 1.5 | 1.5 | 1.5 | 1.5 | 1.5 | 1.5 |
| Water | 12.5 | 12.5 | 12.5 | 12.5 | 12.5 | 12.5 |

### EXAMPLES 76 TO 81

### Preparation of a Microcapsule Carrying [N-methyl-D-alanine³][γ-hydroxy-N-methyl-L-leucine⁴] Cyclosporin A, Compound 3

According to the method described in Formulation 2, microcapsule suspensions carrying the cyclosporin derivative were prepared, with varying compositions of the ingredients shown in Table 12.

TABLE 12
Formulation of microcapsule

| | (unit: weight %) | | | | | |
|---|---|---|---|---|---|---|
| Ingredients | Ex. 76 | Ex. 77 | Ex. 78 | Ex. 79 | Ex. 80 | Ex. 81 |
| Olive oil | 20 | — | 20 | — | 20 | — |
| MCT | — | 20 | — | 20 | — | 20 |
| [N-methyl-D-alanine³][γ-hydroxy-N- | 5 | 5 | 5 | 5 | 5 | 5 |
| methyl-L-leucine⁴] cyclosporin A | | | | | | |
| Gelatin A (75-100 Bloom) | 30 | 30 | — | — | — | — |
| Gelatin A (300 Bloom) | — | — | 30 | 30 | 30 | 30 |
| CMC | 30 | 30 | 30 | 30 | — | — |
| Arabic gum | — | — | — | — | 30 | 30 |
| Acetic acid (10 w % aqueous solution) | 1 | 1 | 1 | 1 | 1 | 1 |
| GA (25 w % aqueous solution) | 1.5 | 1.5 | 1.5 | 1.5 | 1.5 | 1.5 |
| Water | 12.5 | 12.5 | 12.5 | 12.5 | 12.5 | 12.5 |

### EXAMPLES 82 TO 87

### Preparation of a Microcapsule Carrying [γ-hydroxy-N-methyl-L-leucine⁴] Cyclosporin A, Compound 1

According to the method described in Formulation 2, microcapsule suspensions carrying the cyclosporin derivative were prepared, with varying compositions of the ingredients shown in Table 13.

TABLE 13
Formulation of microcapsule

| | (unit: weight %) | | | | | |
|---|---|---|---|---|---|---|
| Ingredients | Ex. 82 | Ex. 83 | Ex. 84 | Ex. 85 | Ex. 86 | Ex. 87 |
| Olive oil | 20 | — | 20 | — | 20 | — |
| MCT | — | 20 | — | 20 | — | 20 |
| [γ-hydroxy-N-methyl-L-leucine⁴] | 0.1 | 0.1 | 0.1 | 0.1 | 0.1 | 0.1 |
| cyclosporin A | | | | | | |
| Gelatin A (75-100 Bloom) | 30 | 30 | — | — | — | — |
| Gelatin A (300 Bloom) | — | — | 30 | 30 | 30 | 30 |
| CMC | 30 | 30 | 30 | 30 | — | — |
| Arabic gum | — | — | — | — | 30 | 30 |
| Acetic acid (10 w % aqueous solution) | 1 | 1 | 1 | 1 | 1 | 1 |
| GA (25 w % aqueous solution) | 1.5 | 1.5 | 1.5 | 1.5 | 1.5 | 1.5 |
| Water | 17.4 | 17.4 | 17.4 | 17.4 | 17.4 | 17.4 |

### EXAMPLES 88 TO 93

### Preparation of a Microcapsule Carrying [γ-hydroxy-N-methyl-L-leucine⁴] Cyclosporin C, Compound 2

According to the method described in Formulation 2, microcapsule suspensions carrying the cyclosporin derivative were prepared, with varying compositions of the ingredients shown in Table 14.

TABLE 14
Formulation of microcapsule

| | (unit: weight %) | | | | | |
|---|---|---|---|---|---|---|
| Ingredients | Ex. 88 | Ex. 89 | Ex. 90 | Ex. 91 | Ex. 92 | Ex. 93 |
| Olive oil | 20 | — | 20 | — | 20 | — |
| MCT | — | 20 | — | 20 | — | 20 |
| [γ-hydroxy-N-methyl-L-leucine⁴] | 0.1 | 0.1 | 0.1 | 0.1 | 0.1 | 0.1 |
| cyclosporin C | | | | | | |
| Gelatin A (75-100 Bloom) | 30 | 30 | — | — | — | — |
| Gelatin A (300 Bloom) | — | — | 30 | 30 | 30 | 30 |
| CMC | 30 | 30 | 30 | 30 | — | — |
| Arabic gum | — | — | — | — | 30 | 30 |
| Acetic acid (10 w % aqueous solution) | 1 | 1 | 1 | 1 | 1 | 1 |
| GA (25 w % aqueous solution) | 1.5 | 1.5 | 1.5 | 1.5 | 1.5 | 1.5 |
| Water | 17.4 | 17.4 | 17.4 | 17.4 | 17.4 | 17.4 |

### EXAMPLES 94 TO 99

### Preparation of a Microcapsule Carrying [N-methyl-D-alanine³][γ-hydroxy-N-methyl-L-leucine⁴] Cyclosporin A, Compound 3

According to the method described in Formulation 2, microcapsule suspensions carrying the cyclosporin derivative were prepared, with varying compositions of the ingredients shown in Table 15.

TABLE 15
Formulation of microcapsule

| | (unit: weight %) | | | | | |
|---|---|---|---|---|---|---|
| Ingredients | Ex. 94 | Ex. 95 | Ex. 96 | Ex. 97 | Ex. 98 | Ex. 99 |
| Olive oil | 20 | — | 20 | — | 20 | — |
| MCT | — | 20 | — | 20 | — | 20 |
| [N-methyl-D-alanine³][γ-hydroxy-N- | 0.1 | 0.1 | 0.1 | 0.1 | 0.1 | 0.1 |
| methyl-L-leucine⁴] cyclosporin A | | | | | | |
| Gelatin A (75-100 Bloom) | 30 | 30 | — | — | — | — |
| Gelatin A (300 Bloom) | — | — | 30 | 30 | 30 | 30 |
| CMC | 30 | 30 | 30 | 30 | — | — |
| Arabic gum | — | — | — | — | 30 | 30 |
| Acetic acid (10 w % aqueous solution) | 1 | 1 | 1 | 1 | 1 | 1 |
| GA (25 w % aqueous solution) | 1.5 | 1.5 | 1.5 | 1.5 | 1.5 | 1.5 |
| Water | 17.4 | 17.4 | 17.4 | 17.4 | 17.4 | 17.4 |

Formulation 3

Preparation of Microsphere Carrying a Cyclosporin Derivative

A cyclosporin derivative, and poly(lactic acid) (PLA, MW 90,000-120,000) (Sigma, USA) or poly(lactic acid-co-glycolic acid) (50:50, MW 50,000-75,000, PLGA) were dissolved in a mixture of chloroform/acetone or dichloromethane/acetone. The solution was added to an aqueous phase of polysorbate 80, and emulsified at 2000 rpm for 10 min, preparing O/W emulsion. The organic solvent was then removed by evaporation at ambient temperature under reduced pressure. After complete evaporation, the residue was added with an aqueous solution at an amount equal to the weight of the solvent removed. A microsphere carrying 5% cyclosporin derivative was thus prepared.

### EXAMPLES 100 TO 103

### Preparation of a Microsphere Carrying [γ-hydroxy-N-methyl-L-leucine⁴] Cyclosporin A, Compound 1

According to the method described in Formulation 3, microsphere suspensions carrying the cyclosporin derivative were prepared, with varying compositions of the ingredients shown in Table 16.

TABLE 16
Formulation of microsphere

| | (unit: weight %) | | | |
|---|---|---|---|---|
| Ingredients | Ex. 100 | Ex. 101 | Ex. 102 | Ex. 103 |
| Chloroform | 30 | 30 | — | — |
| Dichloromethane | — | — | 30 | 30 |
| Acetone | 10 | 10 | 10 | 10 |
| [γ-hydroxy-N-methyl-L-leucine⁴] | 5 | 5 | 5 | 5 |
| cyclosporin A | | | | |
| PLA | 5 | — | 5 | — |
| PLGA | — | 5 | — | 5 |
| Polysorbate 80 | 0.2 | 0.2 | 0.2 | 0.2 |
| Water | 49.8 | 49.8 | 49.8 | 49.8 |

### EXAMPLES 104 TO 107

### Preparation of a Microsphere Carrying [γ-hydroxy-N-methyl-L-leucine⁴] Cyclosporin C, Compound 2

According to the method described in Formulation 3, microsphere suspensions carrying the cyclosporin derivative were prepared, with varying compositions of the ingredients shown in Table 17.

TABLE 17
Formulation of microsphere

| | (unit: weight %) | | | |
|---|---|---|---|---|
| Ingredients | Ex. 104 | Ex. 105 | Ex. 106 | Ex. 107 |
| Chloroform | 30 | 30 | — | — |
| Dichloromethane | — | — | 30 | 30 |
| Acetone | 10 | 10 | 10 | 10 |
| [γ-hydroxy-N-methyl-L-leucine⁴] | 5 | 5 | 5 | 5 |
| cyclosporin C | | | | |
| PLA | 5 | — | 5 | — |
| PLGA | — | 5 | — | 5 |
| Polysorbate 80 | 0.2 | 0.2 | 0.2 | 0.2 |
| Water | 49.8 | 49.8 | 49.8 | 49.8 |

### EXAMPLES 108 TO 111

### Preparation of a Microsphere Carrying [N-methyl-D-alanine³][γ-hydroxy-N-methyl-L-leucine⁴] Cyclosporin A, Compound 3

According to the method described in Formulation 3, microsphere suspensions carrying the cyclosporin derivative were prepared, with varying compositions of the ingredients shown in Table 18.

TABLE 18
Formulation of microsphere

| | (unit: weight %) | | | |
|---|---|---|---|---|
| Ingredients | Ex. 108 | Ex. 109 | Ex. 110 | Ex. 111 |
| Chloroform | 30 | 30 | — | — |
| Dichloromethane | — | — | 30 | 30 |
| Acetone | 10 | 10 | 10 | 10 |
| [N-methyl-D-alanine³][γ-hydroxy- | 5 | 5 | 5 | 5 |
| N-methyl-L-leucine⁴] | | | | |
| cyclosporin A | | | | |
| PLA | 5 | — | 5 | — |
| PLGA | — | 5 | — | 5 |
| Polysorbate 80 | 0.2 | 0.2 | 0.2 | 0.2 |
| Water | 49.8 | 49.8 | 49.8 | 49.8 |

### EXAMPLES 112 TO 115

### Preparation of a Microsphere Carrying [γ-hydroxy-N-methyl-L-leucine⁴] Cyclosporin A, Compound 1

According to the method described in Formulation 3, microsphere suspensions carrying the cyclosporin derivative were prepared, with varying compositions of the ingredients shown in Table 19.

TABLE 19
Formulation of microsphere

| | (unit: weight %) | | | |
|---|---|---|---|---|
| Ingredients | Ex. 112 | Ex. 113 | Ex. 114 | Ex. 115 |
| Chloroform | 30 | 30 | — | — |
| Dichloromethane | — | — | 30 | 30 |
| Acetone | 10 | 10 | 10 | 10 |
| [γ-hydroxy-N-methyl-L-leucine⁴] | 0.1 | 0.1 | 0.1 | 0.1 |
| cyclosporin A | | | | |
| PLA | 5 | — | 5 | — |
| PLGA | — | 5 | — | 5 |
| Polysorbate 80 | 0.2 | 0.2 | 0.2 | 0.2 |
| Water | 54.7 | 54.7 | 54.7 | 54.7 |

### EXAMPLES 116 TO 119

### Preparation of a Microsphere Carrying [γ-hydroxy-N-methyl-L-leucine⁴] Cyclosporin C, Compound 2

According to the method described in Formulation 3, microsphere suspensions carrying the cyclosporin derivative were prepared, with varying compositions of the ingredients shown in Table 20.

TABLE 20
Formulation of microsphere

| | (unit: weight %) | | | |
|---|---|---|---|---|
| Ingredients | Ex. 116 | Ex. 117 | Ex. 118 | Ex. 119 |
| Chloroform | 30 | 30 | — | — |
| Dichloromethane | — | — | 30 | 30 |
| Acetone | 10 | 10 | 10 | 10 |
| [γ-hydroxy-N-methyl-L-leucine⁴] | 0.1 | 0.1 | 0.1 | 0.1 |
| cyclosporin C | | | | |
| PLA | 5 | — | 5 | — |
| PLGA | — | 5 | — | 5 |
| Polysorbate 80 | 0.2 | 0.2 | 0.2 | 0.2 |
| Water | 54.7 | 54.7 | 54.7 | 54.7 |

### EXAMPLES 120 TO 123

### Preparation of a Microsphere Carrying [N-methyl-D-alanine³][γ-hydroxy-N-methyl-L-leucine⁴] Cyclosporin A, Compound 3

According to the method described in Formulation 3, microsphere suspensions carrying the cyclosporin derivative were prepared, with varying compositions of the ingredients shown in Table 21.

TABLE 21
Formulation of microsphere

| | (unit: weight %) | | | |
|---|---|---|---|---|
| Ingredients | Ex. 120 | Ex. 121 | Ex. 122 | Ex. 123 |
| Chloroform | 30 | 30 | — | — |
| Dichloromethane | — | — | 30 | 30 |
| Acetone | 10 | 10 | 10 | 10 |
| [N-methyl-D-alanine³][γ-hydroxy- | 0.1 | 0.1 | 0.1 | 0.1 |
| N-methyl-L-leucine⁴] | | | | |
| cyclosporin A | | | | |
| PLA | 5 | — | 5 | — |
| PLGA | — | 5 | — | 5 |
| Polysorbate 80 | 0.2 | 0.2 | 0.2 | 0.2 |
| Water | 54.7 | 54.7 | 54.7 | 54.7 |

Formulation 4

Preparation of a Cyclosporin/Surfactant Composite Particle

A method of preparing a cyclosporin/surfactant composite particle is as follows. A cyclosporin derivative, and distearyl dimethyl ammonium chloride (DSDAC), sodium lauryl sulfate (SLS), cocodimethyl sulphopropyl betaine (CDSPB), or Tween 60 were mixed in an aqueous phase. The solution was homogenized using an ultra high-pressure dispersion instrument (microfluidizer) at 500 bar, preparing a fine composite particle.

### EXAMPLES 124 TO 127

### Preparation of a [γ-hydroxy-N-methyl-L-leucine⁴] Cyclosporin A, Compound 1/Surfactant Composite Particle

According to the method described in Formulation 4, suspensions of the cyclosporin derivative/surfactant composite particle were prepared, with varying compositions of the ingredients shown in Table 22.

TABLE 22
Formulation of composite particle

| | (unit: weight %) | | | |
|---|---|---|---|---|
| Ingredients | Ex. 124 | Ex. 125 | Ex. 126 | Ex. 127 |
| DSDAC | 1 | — | — | — |
| SLS | — | 1 | — | — |
| CDSPB | — | — | 1 | — |
| Tween 60 | — | — | — | 1 |
| [γ-hydroxy-N-methyl-L-leucine⁴] | 5 | 5 | 5 | 5 |
| cyclosporin A | | | | |
| Water | 94 | 94 | 94 | 94 |

### EXAMPLES 128 TO 131

### Preparation of a [γ-hydroxy-N-methyl-L-leucine⁴] Cyclosporin C, Compound 2/Surfactant Composite Particle

According to the method described in Formulation 4, suspensions of the cyclosporin derivative/surfactant composite particle were prepared, with varying compositions of the ingredients shown in Table 23.

TABLE 23
Formulation of composite particle

| | (unit: weight %) | | | |
|---|---|---|---|---|
| Ingredients | Ex. 128 | Ex. 129 | Ex. 130 | Ex. 131 |
| DSDAC | 1 | — | — | — |
| SLS | — | 1 | — | — |
| CDSPB | — | — | 1 | — |
| Tween 60 | — | — | — | 1 |
| [γ-hydroxy-N-methyl-L-leucine⁴] | 5 | 5 | 5 | 5 |
| cyclosporin C | | | | |
| Water | 94 | 94 | 94 | 94 |

### EXAMPLES 132 TO 135

### Preparation of a [N-methyl-D-alanine³] [γ-hydroxy-N-methyl-L-leucine⁴] Cyclosporin A, Compound 3/Surfactant Composite Particle

According to the method described in Formulation 4, suspensions of the cyclosporin derivative/surfactant composite particle were prepared, with varying compositions of the ingredients shown in Table 24.

TABLE 24
Formulation of composite particle

| | (unit: weight %) | | | |
|---|---|---|---|---|
| Ingredients | Ex. 132 | Ex. 133 | Ex. 134 | Ex. 135 |
| DSDAC | 1 | — | — | — |
| SLS | — | 1 | — | — |
| CDSPB | — | — | 1 | — |
| Tween 60 | — | — | — | 1 |
| [N-methyl-D-alanine³][γ-hydroxy- | 5 | 5 | 5 | 5 |
| N-methyl-L-leucine⁴] | | | | |
| cyclosporin A | | | | |
| Water | 94 | 94 | 94 | 94 |

### EXAMPLES 136 TO 139

### Preparation of a [γ-hydroxy-N-methyl-L-leucine⁴] Cyclosporin A, Compound 1/Surfactant Composite Particle

According to the method described in Formulation 4, suspensions of the cyclosporin derivative/surfactant composite particle were prepared, with varying compositions of the ingredients shown in Table 25.

TABLE 25
Formulation of composite particle

| | (unit: weight %) | | | |
|---|---|---|---|---|
| Ingredients | Ex. 136 | Ex. 137 | Ex. 138 | Ex. 139 |
| DSDAC | 1 | — | — | — |
| SLS | — | 1 | — | — |
| CDSPB | — | — | 1 | — |
| Tween 60 | — | — | — | 1 |
| [γ-hydroxy-N-methyl-L-leucine⁴] | 0.1 | 0.1 | 0.1 | 0.1 |
| cyclosporin A | | | | |
| Water | 98.9 | 98.9 | 98.9 | 98.9 |

### EXAMPLES 140 TO 143

### Preparation of a [γ-hydroxy-N-methyl-L-leucine⁴] Cyclosporin C, Compound 2/Surfactant Composite Particle

According to the method described in Formulation 4, suspensions of the cyclosporin derivative/surfactant composite particle were prepared, with varying compositions of the ingredients shown in Table 26.

TABLE 26
Formulation of composite particle

| | (unit: weight %) | | | |
|---|---|---|---|---|
| Ingredients | Ex. 140 | Ex. 141 | Ex. 142 | Ex. 143 |
| DSDAC | 1 | — | — | — |
| SLS | — | 1 | — | — |
| CDSPB | — | — | 1 | — |
| Tween 60 | — | — | — | 1 |
| [γ-hydroxy-N-methyl-L-leucine⁴] | 0.1 | 0.1 | 0.1 | 0.1 |
| cyclosporin C | | | | |
| Water | 98.9 | 98.9 | 98.9 | 98.9 |

### EXAMPLES 144 TO 147

### Preparation of a [N-methyl-D-alanine³] [γ-hydroxy-N-methyl-L-leucine⁴] Cyclosporin A, Compound 3/Surfactant Composite Particle

According to the method described in Formulation 4, suspensions of the cyclosporin derivative/surfactant composite particle were prepared, with varying compositions of the ingredients shown in Table 27.

TABLE 27
Formulation of composite particle

| | (unit: weight %) | | | |
|---|---|---|---|---|
| Ingredients | Ex. 144 | Ex. 145 | Ex. 146 | Ex. 147 |
| DSDAC | 1 | — | — | — |
| SLS | — | 1 | — | — |
| CDSPB | — | — | 1 | — |
| Tween 60 | — | — | — | 1 |
| [N-methyl-D-alanine³][γ-hydroxy- | 0.1 | 0.1 | 0.1 | 0.1 |
| N-methyl-L-leucine⁴] | | | | |
| cyclosporin A | | | | |
| Water | 98.9 | 98.9 | 98.9 | 98.9 |

Formulation 5

Preparation of an Emulsion Containing a Cyclosporin Derivative

A method of preparing an emulsion containing a cyclosporin derivative is as follows. To a mixture of olive oil, triglyceride (C₁₀-C₁₈, TG) and octylsalicylate (OS), a cyclosporin derivative was added and dissolved. The solution was added to an aqueous polyvinyl alcohol (PVA, MW 30,000-70,000) (Sigma, USA) solution or an aqueous polysorbate 20 solution. The resulting solution was emulsified at 2000 rpm for 10 min at ambient temperature.

### EXAMPLES 148 TO 153

### Preparation of an Emulsion Containing [γ-hydroxy-N-methyl-L-leucine⁴] Cyclosporin A, Compound 1

According to the method described in Formulation 5, emulsions containing the cyclosporin derivative were prepared, with varying compositions of the ingredients shown in Table 28.

TABLE 28
Formulation of emulsion

| | (unit: weight %) | | | | | |
|---|---|---|---|---|---|---|
| Ingredients | Ex. 148 | Ex. 149 | Ex. 150 | Ex. 151 | Ex. 152 | Ex. 153 |
| Olive oil | 30 | 30 | — | — | — | — |
| MCT | — | — | 30 | 30 | — | — |
| OS | — | — | — | — | 30 | 30 |
| [γ-hydroxy-N-methyl-L-leucine⁴] | 5 | 5 | 5 | 5 | 5 | 5 |
| cyclosporin A | | | | | | |
| PVA | 0.5 | — | 0.5 | — | 0.5 | — |
| polysorbate 20 | — | 0.5 | — | 0.5 | — | 0.5 |
| Water | 64.5 | 64.5 | 64.5 | 64.5 | 64.5 | 64.5 |

### EXAMPLES 154 TO 159

### Preparation of an Emulsion Containing [γ-hydroxy-N-methyl-L-leucine⁴] Cyclosporin C, Compound 2

According to the method described in Formulation 5, emulsions containing the cyclosporin derivative were prepared, with varying compositions of the ingredients shown in Table 29.

TABLE 29
Formulation of emulsion

| | (unit: weight %) | | | | | |
|---|---|---|---|---|---|---|
| Ingredients | Ex. 154 | Ex. 155 | Ex. 156 | Ex. 157 | Ex. 158 | Ex. 159 |
| Olive oil | 30 | 30 | — | — | — | — |
| MCT | — | — | 30 | 30 | — | — |
| OS | — | — | — | — | 30 | 30 |
| [γ-hydroxy-N-methyl-L-leucine⁴] | 5 | 5 | 5 | 5 | 5 | 5 |
| cyclosporin C | | | | | | |
| PVA | 0.5 | — | 0.5 | — | 0.5 | — |
| polysorbate 20 | — | 0.5 | — | 0.5 | — | 0.5 |
| Water | 64.5 | 64.5 | 64.5 | 64.5 | 64.5 | 64.5 |

### EXAMPLES 160 TO 165

### Preparation of an Emulsion Containing [N-methyl-D-alanine³] [γ-hydroxy-N-methyl-L-leucine⁴] Cyclosporin A, Compound 3

According to the method described in Formulation 5, emulsions containing the cyclosporin derivative were prepared, with varying compositions of the ingredients shown in Table 30.

TABLE 30
Formulation of emulsion

| | (unit: weight %) | | | | | |
|---|---|---|---|---|---|---|
| Ingredients | Ex. 160 | Ex. 161 | Ex. 162 | Ex. 163 | Ex. 164 | Ex. 165 |
| Olive oil | 30 | 30 | — | — | — | — |
| MCT | — | — | 30 | 30 | — | — |
| OS | — | — | — | — | 30 | 30 |
| [N-methyl-D-alanine³] [γ-hydroxy- | 5 | 5 | 5 | 5 | 5 | 5 |
| N-methyl-L-leucine⁴] cyclosporin A | | | | | | |
| PVA | 0.5 | — | 0.5 | — | 0.5 | — |
| polysorbate 20 | — | 0.5 | — | 0.5 | — | 0.5 |
| Water | 64.5 | 64.5 | 64.5 | 64.5 | 64.5 | 64.5 |

### EXAMPLES 166 TO 171

### Preparation of an Emulsion Containing [γ-hydroxy-N-methyl-L-leucine⁴] Cyclosporin A, Compound 1

According to the method described in Formulation 5, emulsions containing the cyclosporin derivative were prepared, with varying compositions of the ingredients shown in Table 31.

TABLE 31
Formulation of emulsion

| | (unit: weight %) | | | | | |
|---|---|---|---|---|---|---|
| Ingredients | Ex. 166 | Ex. 167 | Ex. 168 | Ex. 169 | Ex. 170 | Ex. 171 |
| Olive oil | 30 | 30 | — | — | — | — |
| MCT | — | — | 30 | 30 | — | — |
| OS | — | — | — | — | 30 | 30 |
| [γ-hydroxy-N-methyl-L-leucine⁴] | 0.1 | 0.1 | 0.1 | 0.1 | 0.1 | 0.1 |
| cyclosporin A | | | | | | |
| PVA | 0.5 | — | 0.5 | — | 0.5 | — |
| polysorbate 20 | — | 0.5 | — | 0.5 | — | 0.5 |
| Water | 69.4 | 69.4 | 69.4 | 69.4 | 69.4 | 69.4 |

### EXAMPLES 172 TO 177

### Preparation of an Emulsion Containing [γ-hydroxy-N-methyl-L-leucine⁴] Cyclosporin C, Compound 2

According to the method described in Formulation 5, emulsions containing the cyclosporin derivative were prepared, with varying compositions of the ingredients shown in Table 32.

TABLE 32
Formulation of emulsion

| | (unit: weight %) | | | | | |
|---|---|---|---|---|---|---|
| Ingredients | Ex. 172 | Ex. 173 | Ex. 174 | Ex. 175 | Ex. 176 | Ex. 177 |
| Olive oil | 30 | 30 | — | — | — | — |
| MCT | — | — | 30 | 30 | — | — |
| OS | — | — | — | — | 30 | 30 |
| [γ-hydroxy-N-methyl-L-leucine⁴] | 0.1 | 0.1 | 0.1 | 0.1 | 0.1 | 0..1 |
| cyclosporin C | | | | | | |
| PVA | 0.5 | — | 0.5 | — | 0.5 | — |
| polysorbate 20 | — | 0.5 | — | 0.5 | — | 0.5 |
| Water | 69.4 | 69.4 | 69.4 | 69.4 | 69.4 | 69.4 |

### EXAMPLES 178 TO 183

### Preparation of an Emulsion Containing [N-methyl-D-alanine³][γ-hydroxy-N-methyl-L-leucine⁴] Cyclosporin A, Compound 3

According to the method described in Formulation 5, emulsions containing the cyclosporin derivative were prepared, with varying compositions of the ingredients shown in Table 33.

TABLE 33
Formulation of emulsion

| | (unit: weight %) | | | | | |
|---|---|---|---|---|---|---|
| Ingredients | Ex. 178 | Ex. 179 | Ex. 180 | Ex. 181 | Ex. 182 | Ex. 183 |
| Olive oil | 30 | 30 | — | — | — | — |
| MCT | — | — | 30 | 30 | — | — |
| OS | — | — | — | — | 30 | 30 |
| [N-methyl-D-alanine³][γ-hydroxy- | 0.1 | 0.1 | 0.1 | 0.1 | 0.1 | 0..1 |
| N-methyl-L-leucine⁴] cyclosporin A | | | | | | |
| PVA | 0.5 | — | 0.5 | — | 0.5 | — |
| polysorbate 20 | — | 0.5 | — | 0.5 | — | 0.5 |
| Water | 69.4 | 69.4 | 69.4 | 69.4 | 69.4 | 69.4 |

### TEST EXAMPLE 1

Measurements of Size and Zeta Potential

With respect to representative particles carrying a cyclosporin derivative prepared in Examples, their sizes and zeta potentials were measured using Zeta Plus instrument (Brookhaven Instruments Co.). The results are shown in Table 34.

TABLE 34
Properties of carriers of the invention

| Carrier | Average size (μm) | Zeta potential (mV) |
|---|---|---|
| Liposome of Ex. 1 | 6.0 | −20.1 |
| Liposome of Ex. 3 | 5.4 | −22.3 |
| Liposome of Ex. 5 | 5.7 | +43.4 |
| Liposome of Ex. 7 | 6.3 | +33.0 |
| Microcapsule of Ex. 65 | 6.6 | −32.2 |
| Microcapsule of Ex. 67 | 5.5 | −30.5 |
| Microcapsule of Ex. 69 | 6.0 | −28.4 |
| Microsphere of Ex. 101 | 5.3 | −15.3 |
| Microsphere of Ex. 103 | 5.4 | −17.2 |
| Composite particle of Ex. 124 | 6.2 | +44.1 |
| Composite particle of Ex. 125 | 7.2 | −30.1 |
| Emulsion of Ex. 149 | 6.9 | −20.6 |
| Emulsion of Ex. 151 | 7.1 | −18.5 |
| Emulsion of Ex. 153 | 7.2 | −17.7 |

It can been seen in Table 34 that as for the composite particles, their sizes were approximately 5 to 7 μm. The respective zeta potentials indicate surface properties of the composite particles. For example, the cationic composite particle the example 124 showed a plus (+) zeta potential value, while the negative composite particle, the particle of the example 125 showed a minus (−) zeta potential value.

### TEST EXAMPLE 2

Evaluation of Skin Penetration In Vitro

Female hairless SKH1 mice of ages 6 to 8 weeks were employed. To measure in vitro skin absorption of the carrier particles of the invention, a diffusion cell consisting of a donor chamber and a receptor chamber was utilized. The mice skin was positioned between the two chambers, in which the epidermis was directed to the donor chamber while the dermis was directed to the receptor chamber. Phosphate-buffered saline (pH 7.4, 37° C.) was filled in the receptor chamber and let stand for 1 hr, thereby the skin and the buffer solution reaching equilibrium. Then, 300 mg of the carrier particle suspensions (in a suspension, a cyclosporin content is 5%) were respectively applied to the epidermis (the applied area is 3.14 cm²). The donor chambers were sealed with parafilm. After 12 hrs, a 0.2 ml fluid was sampled from the receptor chamber, and the amount of the cyclosporin derivative penetrated through the skin was quantified by means of HPLC. The data are shown in Table 35.

TABLE 35
Skin absorption profile

| | Amount of cyclosporin |
|---|---|
| Carrier | penetrated (mg) |
| Liposome of Ex. 1 | 3.78 |
| Liposome of Ex. 3 | 3.66 |
| Liposome of Ex. 5 | 3.89 |
| Liposome of Ex. 7 | 3.63 |
| Microcapsule of Ex. 65 | 2.55 |
| Microcapsule of Ex. 67 | 2.87 |
| Microcapsule of Ex. 69 | 2.78 |
| Microsphere of Ex. 101 | 3.10 |
| Microsphere of Ex. 103 | 3.30 |
| Composite particle of Ex. 124 | 2.88 |
| Composite particle of Ex. 125 | 2.98 |
| Emulsion of Ex. 149 | 3.44 |
| Emulsion of Ex. 151 | 2.99 |
| Emulsion of Ex. 153 | 3.01 |
| 5% [γ-hydroxy-N-methyl-L-leucine⁴] | 1.23 |
| cyclosporin A in ethanol | |
| 5% [γ-hydroxy-N-methyl-L-leucine⁴] | 0.97 |
| cyclosporin A in acetone | |

As shown in Table 35, the fine carrier particles of Examples, which are fine particles of several microns in size, showed skin penetration of cyclosporin derivatives 2 to 3 times higher than those of free cyclosporin derivatives dissolved in ethanol or acetone solutions, upon application to mouse skin. Thus, the carrier particles of the invention such as liposomes, microcapsules, microspheres, composite particles and emulsions, in which a cyclosporin derivative is encapsulated, have an advantage of higher skin penetration than those of free cyclosporin derivatives and dissolved in organic solvent at a molecular level.

### TEST EXAMPLE 3

Evaluation of Hair Growth Promoting Effect by Carriers Loading [γ-hydroxy-N-methyl-L-leucine⁴] Cyclosporin A, Compound 1

Female C57BL/6 mice of ages 6 to 7 weeks were utilized. After removing hairs on the middle of the back with an electric shaver, the mice were weighed and randomly assigned to the test groups with an even distribution of weights. After one day of adaptation, the mice were applied with respective carrier suspensions loading 0.1% [γ-hydroxy-N-methyl-L-leucine⁴] cyclosporin A to their hair removed areas once a day at a dose of 100 μl, for 30 days. The results were determined by visual examination, in terms of degrees of hair regrowth. With respect to respective hair-removed areas, rates of new hair growth were examined and compared.

As can be seen in Table 36, the carrier particles of the invention such as liposomes, microcapsules, microspheres, composite particles and emulsions, in which the cyclosporin derivative is encapsulated, the particles being fine particles of several microns in size, showed a significant hair growth promoting effect, owing to their higher penetration to the skin and follicle, than that of the free cyclosporin derivative in organic solvent such as 50% ethanol. Meanwhile, over the course of 30 days, comparing the appearance of the backs, the mice of the control and all test groups showed no specific skin irritation.

TABLE 36
Hair regrowth in mice

| Carrier applied | Area rate of hair regrowth (%) |
|---|---|
| Liposome of Ex. 43 | 90 |
| Liposome of Ex. 45 | 91 |
| Liposome of Ex. 47 | 89 |
| Liposome of Ex. 49 | 85 |
| Microcapsule of Ex. 82 | 86 |
| Microcapsule of Ex. 84 | 90 |
| Microcapsule of Ex. 86 | 91 |
| Microsphere of Ex. 112 | 91 |
| Microsphere of Ex. 114 | 93 |
| Composite particle of Ex. 136 | 91 |
| Composite particle of Ex. 138 | 88 |
| Emulsion of Ex. 166 | 90 |
| Emulsion of Ex. 168 | 92 |
| Emulsion of Ex. 170 | 86 |
| 0.1% [γ-hydroxy-N-methyl-L- | 62 |
| leucine⁴] cyclosporin A in | |
| a 50% aqueous ethanol solution | |

### EXAMPLES 184 TO 188 AND COMPARATIVE EXAMPLE 2

Use for Formulation of Shampoo

The liposomes, microcapsules, microspheres, composite particles and emulsions, prepared in the above Examples, and powder of the cyclosporin derivative which is not formulated, were respectively added to compositions for hair use. The mixtures were blended at about 35 to 40° C. in a stirrer (300 to 400 rpm), preparing respective homogeneous hair cleaning compositions (shampoos). The content of the cyclosporin derivative was set to 1% in the shampoo composition. Table 37 shows the contents of individual ingredients.

TABLE 37
Formulation of shampoo

| | (unit: weight %) | | | | | |
|---|---|---|---|---|---|---|
| | | | | | | Comp. |
| Ingredients | Ex. 184 | Ex. 185 | Ex. 186 | Ex. 187 | Ex. 188 | Ex. 2 |
| Hair cleaning Composition | | | | | | |
| Water | 53 | 53 | 53 | 53 | 53 | 72 |
| Betaine | 1 | 1 | 1 | 1 | 1 | 1 |
| SLES | 20 | 20 | 20 | 20 | 20 | 20 |
| SLS | 5 | 5 | 5 | 5 | 5 | 5 |
| CDE | 1 | 1 | 1 | 1 | 1 | 1 |
| Liposome of Ex. 1 | | | | | | |
| (Compound 1: 5% content) | 20 | — | — | — | — | — |
| Microcapsule of Ex. 65 | | | | | | |
| (Compound 1: 5% content) | — | 20 | — | — | — | — |
| Microsphere of Ex. 101 | | | | | | |
| (Compound 1: 5% content) | — | — | 20 | — | — | — |
| Composite particle of Ex. 124 | | | | | | |
| (Compound 1: 5% content) | — | — | — | 20 | — | — |
| Emulsion of Ex. 149 | | | | | | |
| (Compound 1: 5% content) | — | — | — | — | 20 | — |
| Powder of cyclosporin derivative | | | | | | |
| (Compound 1: 100% content) | — | — | — | — | — | 1 |

Note:
Betaine used herein is cocamidopropyl betaine;
SLES used herein is sodium lauryl ether sulfate, which has two ethylene oxide groups and 12 carbon atoms;
SLS used herein is sodium lauryl sulfate, which has 12 carbon atoms;
CDE represents coconut diethanolamide; and in the first column, each of the contents of cyclosporin parenthesized represents the content of cyclosporin derivative carried in each formulation.

### TEST EXAMPLE 4

Test of Shampoo for Phase Stability Over Time

To evaluate phase stability of hair cleaning preparations over an elapsed time, the shampoos of Examples 184 to 188 and Comparative Example 2 were stored for 90 days at ambient temperature and at 40° C., respectively. Properties of the shampoos were observed, and the results are shown in Table 38.

TABLE 38
Phase stability of shampoo

| | Ex. | Ex. | Ex. | Ex. | Ex. | |
|---|---|---|---|---|---|---|
| Temperature | 184 | 185 | 186 | 187 | 188 | Comp. Ex. 2 |
| ambient temp. | stable | stable | stable | stable | stable | precipitated |
| 40° C. | stable | stable | stable | stable | stable | precipitated |

As can be seen in Table 38, the carrier particles of the invention such as liposomes, microcapsules, microspheres, composite particles and emulsions, in which the cyclosporin derivative is encapsulated, showed good phase stability over time. On the other hand, the powder of cyclosporin derivative which is not formulated was unstable in the hair cleaning composition. Thus, there is another advantage in that the carrier particles of the invention in which the cyclosporin derivative is encapsulated, can protect the free cyclosporin derivative from being phase conversion over time, in shampoo compositions.

### EXAMPLES 189 TO 193 AND COMPARATIVE EXAMPLE 3

Use for Formulation of Rinse

The liposomes, microcapsules, microspheres, composite particles and emulsions, prepared in the above Examples, and powder of the cyclosporin derivative which is not formulated, were respectively added to rinse compositions. The mixtures were blended at about 35 to 40° C. in a stirrer (200 to 300 rpm), preparing respective homogeneous compositions (rinses). Table 39 shows the contents of individual ingredients.

TABLE 39
Formulation of rinse

| | (unit: weight %) | | | | | |
|---|---|---|---|---|---|---|
| | | | | | | Comp. |
| Ingredients | Ex. 189 | Ex. 190 | Ex. 191 | Ex. 192 | Ex. 193 | Ex. 3 |
| Rinse Composition | | | | | | |
| Behenyl trimethyl | 0.3 | 0.3 | 0.3 | 0.3 | 0.3 | 0.3 |
| ammonium chloride | | | | | | |
| Liquid paraffin | 1.5 | 1.5 | 1.5 | 1.5 | 1.5 | 1.5 |
| Stearyl alcohol | 5 | 5 | 5 | 5 | 5 | 5 |
| Glycerin | 1 | 1 | 1 | 1 | 1 | 1 |
| Cetyl trimethyl | 5 | 5 | 5 | 5 | 5 | 5 |
| ammonium chloride | | | | | | |
| Silicone | 2 | 2 | 2 | 2 | 2 | 2 |
| Water | 65.2 | 65.2 | 65.2 | 65.2 | 65.2 | 84.2 |
| Liposome of Ex. 1 | | | | | | |
| (Compound 1: 5% content) | 20 | — | — | — | — | — |
| Microcapsule of Ex. 65 | | | | | | |
| (Compound 1: 5% content) | — | 20 | — | — | — | — |
| Microsphere of Ex. 101 | | | | | | |
| (Compound 1: 5% content) | — | — | 20 | — | — | — |
| Composite particle of Ex. 124 | | | | | | |
| (Compound 1: 5% content) | — | — | — | 20 | — | — |
| Emulsion of Ex. 149 | | | | | | |
| (Compound 1: 5% content) | — | — | — | — | 20 | — |
| Powder of cyclosporin derivative | | | | | | |
| (Compound 1: 100% content) | — | — | — | — | — | 1 |

Note:
Silicone used herein is a silicone oil emulsion (Dow Corning Co., 2-1691 emulsion); and in the first column, each of the contents of cyclosporin parenthesized represents the content of cyclosporin derivative carried in each formulation.

### TEST EXAMPLE 5

Test of Rinse for Phase Stability Over Time

To evaluate phase stability of rinse compositions with an elapsed time, the rinses of Examples 189 to 193 and Comparative Example 3 were stored for 90 days at ambient temperature and at 40° C., respectively. Properties of the rinses were observed, and the results are shown in Table 40.

TABLE 40
Phase stability of rinse

| | Ex. | Ex. | Ex. | Ex. | Ex. | |
|---|---|---|---|---|---|---|
| Temperature | 189 | 190 | 191 | 192 | 193 | Comp. Ex. 3 |
| ambient temp. | stable | stable | stable | stable | stable | precipitated |
| 40° C. | stable | stable | stable | stable | stable | precipitated |

As can be seen in Table 40, the carrier particles of the invention such as liposomes, microcapsules, microspheres, composite particles and emulsions, in which the cyclosporin derivative is encapsulated, showed good phase stability over time. On the other hand, the powder of cyclosporin derivative which is not formulated, was unstable in the rinses. Thus, there is another advantage in that the carrier particles of the invention in which the cyclosporin derivative is encapsulated, can protect the cyclosporin derivative from phase conversion over time, in rinse compositions.

### TEST EXAMPLE 6

Evaluation of Hair Restoring Effect by Shampoo and Rinse Containing Carriers Loading [γ-hydroxy-N-methyl-L-leucine⁴] Cyclosporin A, Compound 1

Female C57BL/6 mice of ages 6 to 7 weeks were utilized. After removing hairs on the middle of the back with an electric shaver, the mice were weighed and randomly assigned to the test groups with an even distribution of weights. After one day of adaptation, the mice were applied with respective samples of the shampoos or rinses (content of each cyclosporin derivative was 1.0%) prepared above at amounts of 200 μl to their hair removed areas, and 3 min later, the applied areas were washed with running water for 10 sec. Starting from the day after hair removal, the treatment of the samples was carried out 5 to 6 times per week, once a day for 30 days. On day 30, after the experiment begins, the experimental animals were anesthetized with phentobarbitol, and photographed.

The hair restoring effects were determined, based on the areas in which hairs were restored. The hair restoration rates were calculated using the area in which the hair restoring effect was shown, measured on the 30^{th} day after treatment, with respect to respective hair-removed areas immediately after hair removal. The results are shown in Table 41.

TABLE 41
Hair restore in mice

| | | Hair | | Hair |
|---|---|---|---|---|
| Carrier used | Shampoo | growth | Rinse | growth |
| Liposome | Ex. 184 | +++ | Ex. 189 | +++ |
| Microcapsule | Ex. 185 | +++ | Ex. 190 | +++ |
| Microsphere | Ex. 186 | +++ | Ex. 191 | +++ |
| Composite Paricle | Ex. 187 | +++ | Ex. 192 | +++ |
| Emulsion | Ex. 188 | ++ | Ex. 193 | +++ |
| Cyclosporin powder | Comp. Ex. 2 | + | Comp. Ex. 3 | + |
| (not formulated) | | | | |

Evaluations are as follows:
−, The hair restoration rate is 10% or less;
+, The rate is 10-30%;
++, The rate is 30-70%; and
+++, The rate is 70-100%.

As can be seen in Table 41, the shampoos and rinses containing the carrier particles of the invention such as liposomes, microcapsules, microspheres, composite particles and emulsions, in which the cyclosporin derivative is encapsulated, showed a hair restoring effect higher than those of the shampoo or rinse which contain the free cyclosporin derivative.

### Industrial Applicability

As apparent from the above description, the present invention provides topical scalp and transdermal preparations comprising a [γ-hydroxy-N-methyl-L-leucine⁴] cylosporin which is non-immunosuppressive. The preparations of the invention show a high degree of penetration to the skin and follicle, thus capable of being employed as a hair-restoring agent and applied for the prevention of hair loss.

1. A topical scalp and transdermal preparation with good penetration to the skin and follicle, prepared by encapsulating a [γ-hydroxy-N-methyl-L-leucine⁴] cylosporin derivative, represented by Chemical Formula 1 below, having an excellent hair restoring effect without immunosuppresive activity, in a carrier selected from the group consisting of a liposome, microcapsule, microsphere, composite particle and emulsion: 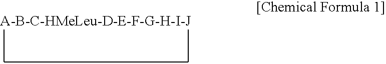
in which
A represents N-methyl-(4R)-4-[(E)-2-butenyl]-4-methyl-L-threonine, (2S,3R,4R,6E)-3-sulfhydryl-4-methyl-2-(methylamino)-6-octenoic acid or (2S,4R,6E)-3-oxo-4-methyl-2-(methylamino)-6-octenoic acid;
B represents L-α-aminobutyric acid (Abu), L-alanine (Ala), L-threonine (Thr), L-valine (Val) or L-norvaline (Nva);
C represents a D-amino acid represented by the general formula (“1”), CH₃NH—CH(R)—COOH in which,
R is one selected from the group consisting of hydrogen, C₁-C₆ straight or branched alkyl, alkenyl or alkynyl moieties, substituted or unsubstituted, with one or more moieties selected from the group consisting of amino, hydroxy, halo, haloalkyl, ester, alkoxy, cyano, nitro, alkylamino, and dialkylamino, and general formula (“2”) X—R′ in which,
X is oxygen or sulfur, and
R′ is one selected from the group consisting of hydrogen, and C₁-C₆ straight or branched alkyl, alkenyl or alkynyl moieties, substituted or unsubstituted, with one or more moieties selected from the group consisting of amino, hydroxy, halo, haloalkyl, ester, alkoxy, cyano, nitro, alkylamino, and dialkylamino;
HMeLeu represents γ-hydroxy N-methyl-L-leucine;
D represents L-valine, L-norvaline or L-leucine;
E represents N-methyl-L-leucine, γ-hydroxy N-methyl-L-leucine, or L-leucine;
F represents L-alanine or L-alanine thioamide ([⁷ψ⁸ CS—NH], NH—CHCH₃—CS—);
G represents D-hydroxyisovaleric acid or a D-amino acid represented by the general formula (“3), —NH—CH(CH₂R)—COOH in which,
R is hydrogen or general formula (“4”) X—R′ in which,
X is oxygen or sulfur, and
R′ is one selected from the group consisting of hydrogen, and C₁-C₆ straight or branched alkyl, alkenyl or alkynyl moieties, substituted or unsubstituted, with one or more moieties selected from the group consisting of amino, hydroxy, halo, haloalkyl, ester, alkoxy, cyano, nitro, alkylamino, and dialkylamino;
H represents N-methyl-L-leucine, y-hydroxy-N-methyl-L-leucine or L-leucine;
I represents N-methyl-L-leucine, y-hydroxy-N-methyl-L-leucine or L-leucine; and
J represents N-methyl-L-valine or L-valine.

2. The topical scalp and transdermal preparation as set forth in claim 1, wherein the cyclosporin derivative is represented by Chemical Formula 2: 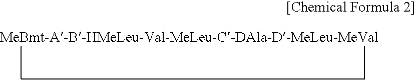
in which
MeBmt represents N-methyl-(4R)-4-[(E)-2-butenyl]-4-methyl-L-threonine;
A′ represents L-α-aminobutyric acid, L-alanine, L-threonine, L-valine or L-norvaline;
B′ represents N-methyl-D-alanine, D-2-(methylamino)pent-4-enoyl, N-methyl-D-aminobutyric acid, N-methyl-D-norvaline, D-2-(methylamino)hexa-4-ynoyl, D-2-(methylamino)pent-4-ynoyl, D-2-methylthio-sarcosine, N-methyl-O-propenyl-D-serine or N-methyl-D-serine;
HMeLeu represents γ-hydroxy-N-methyl-L-leucine;
Val represents L-valine;
MeLeu represents N-methyl-L-leucine;
C′ represents L-alanine or L-alanine thioamide ([⁷ψ⁸ CS—NH], NH—CHCH₃—CS—);
DAla represents D-alanine;
D′ represents N-methyl-L-leucine, γ-hydroxy-N-methyl-L-leucine or L-leucine; and
MeVal represents N-methyl-L-valine.

3. The topical scalp and transdermal preparation as set forth in claim 1, wherein the cyclosporin derivative is represented by Chemical Formula 3: 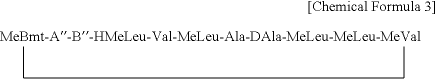
in which
MeBmt represents N-methyl-(4R)-4-[(E)-2-butenyl]-4-methyl-L-threonine;
A″ represents L-α-aminobutyric acid, L-alanine, L-threonine, L-valine or L-norvaline;
B″ represents N-methyl-D-alanine, D-2-(methylamino)pent-4-enoyl, N-methyl-D-aminobutyric acid, N-methyl-D-norvaline, D-2-(methylamino)hexa-4-ynoyl, D-2-(methylamino)pent-4-ynoyl, D-2-methylthio-sarcosine, N-methyl-O-propenyl-D-serine or N-methyl-D-serine;
HMeLeu represents γ-hydroxy-N-methyl-L-leucine;
Val represents L-valine;
Ala represents L-alanine;
MeLeu represents N-methyl-L-leucine;
DAla represents D-alanine; and
MeVal represents N-methyl-L-valine.

4. The topical scalp and transdermal preparation as set forth in claim 1, wherein the cyclosporin derivative is [γ-hydroxy-N-methyl-L-leucine⁴] cylosporin A.

5. The topical scalp and transdermal preparation as set forth in claim 1, wherein the cyclosporin derivative is [γ-hydroxy-N-methyl-L-leucine⁴] cylosporin C.

6. The topical scalp and transdermal preparation as set forth in claim 1, wherein the cyclosporin derivative is [N-methyl-D-alanine³] [γ-hydroxy-N-methyl-L-leucine⁴] cyclosporin A.

7. The topical scalp and transdermal preparation as set forth in claim 1, wherein the cyclosporin derivative is [γ-hydroxy-N-methyl-L-leucine⁴] [γ-hydroxy-N-methyl-L-leucine⁹] cyclosporin A.

8. The topical scalp and transdermal preparation as set forth in claim 1, wherein the cyclosporin derivative is [γ-hydroxy-N-methyl-L-leucine⁴] [alanine thiomide⁷] cyclosporin A (or [⁷ψ⁸ CS—NH] cyclosporin A).

9. The topical scalp and transdermal preparation as set forth in claim 1, wherein the cyclosporin derivative is [L-threonine]²[L-leucine]⁵[γ-hydroxy-N-methyl-L-leucine⁴] [D-hydroxyisovaleric acid]⁸[L-leucine]¹⁰ cyclosporin A.

10. The topical scalp and transdermal preparation as set forth in claim 1, wherein the cyclosporin derivative is [γ-hydroxy-N-methyl-L-leucine⁴] [D-serine] cyclosporin A.

11. The topical scalp and transdermal preparation as set forth in claim 1, wherein the liposome encapsulating the cyclosporin derivative is prepared by the steps of:
dissolving amphiphilic molecules and the cyclosporin derivative in organic solvent;
evaporating the organic solvent at ambient temperature, thereby giving a mixture of dry lipid film consisting of the amphiphilic molecules and the cyclosporin derivative;
hydrating the dry phospholipid film by adding a certain amount of an aqueous solution; and
homogenizing the resultant film using a mechanical dispersion instrument.

12. The topical scalp and transdermal preparation as set forth in claim 11, wherein the organic solvent is selected from the group consisting of acetone, methanol, ethanol, isopropanol, chloroform and dichloromethane.

13. The topical scalp and transdermal preparation as set forth in claim 11, wherein a weight ratio of the cyclosporin derivative/the amphiphilic molecules is 1/100 to 1/1.

14. The topical scalp and transdermal preparation as set forth in claim 11, wherein the mechanical dispersion instrument is selected from the group consisting of a colloid mill, a roll mill, a sonicator, a high-pressure dispersion instrument (microfluidizer, Microfluidics Corp., USA), Ultra Turrax (Janke and Kunkel, Germany), Nanoget (Nanoget Engineering, Germany) and Brogli (Italy).

15. The topical scalp and transdermal preparation as set forth in claim 11, wherein the amphiphilic molecules are selected from the group consisting of phospholipids such as phosphatidylcholine (PC), phosphatidylethanolamine (PE) and phosphatidylinositol (PI), cholesterol, cationic surfactants such as stearamidopropyldimethylamine (SAPDA), distearyldimethylammoniumchloride (DSDAC) and stearyltrimethylammoniumchloride (STAC), and stearic acid (SA).

16. The topical scalp and transdermal preparation as set forth in claim 11, wherein the liposome has a diameter of several nanometers to several micrometers.

17. The topical scalp and transdermal preparation as set forth in claim 1, wherein the microcapsules encapsulating the cyclosporin derivative is prepared by the steps of:
dissolving the cyclosporin derivative in an oil phase;
emulsifying the oil phase in an aqueous solution;
creating a chemical reaction of capsule wall materials in the aqueous phase of the emulsion.

18. The topical scalp and transdermal preparation as set forth in claim 17, wherein the capsule wall materials are selected from the group consisting of gelatin, carboxymethyl cellulose, arabic gum, acacia gum, methylol melamine, and methylol urea.

19. The topical scalp and transdermal preparation as set forth in claim 17, wherein the capsule wall materials in the aqueous phase of the emulsion are subjected to condensation reaction.

20. The topical scalp and transdermal preparation as set forth in any one of claim 1, wherein the microcapsule encapsulating the cyclosporin derivative is prepared by the steps of:
dissolving the cyclosporin derivative and a polymer in an oil phase;
dispersing the oil phase in a second immiscible phase; and
evaporating the oil phase.

21. The topical scalp and transdermal preparation as set forth in claim 20, wherein the polymer is selected from the group consisting of poly(lactic acid) (PLA), poly(lactic acid-co-glycolic acid) (PLGA), poly(ε-caprolactone) (PECL), and cellulose-acetate phthalate.

22. The topical scalp and transdermal preparation as set forth in claim 20, wherein the oil phase is selected from the group consisting of chloroform, dichloromethane, a mixture of dichloromethane/acetone, and a mixture of dichloromethane/acetone.

23. The topical scalp and transdermal preparation as set forth in claim 20, wherein the second phase immiscible with the oil phase is an aqueous phase or gas phase.

24. The topical scalp and transdermal preparation as set forth in claim 1, wherein the composite particle encapsulating the cyclosporin derivative is prepared by the steps of:
mixing the cyclosporin derivative and surfactant in an aqueous phase; and
forcibly dispersing the solution using a mechanical dispersion instrument.

25. The topical scalp and transdermal preparation as set forth in claim 24, wherein the surfactant is selected from the group consisting of cationic surfactants such as distearyl dimethyl ammonium chloride (DSDAC), anionic surfactants such as sodium lauryl sulfate (SLS), amphiphlic surfactants such as cocodimethyl sulphopropyl betaine (CDSPB), and nonionic surfactants such as Tween 60.

26. The topical scalp and transdermal preparation as set forth in claim 24, wherein the cyclosporin derivative is contained at 0.01 to 35 weight % in a mixture of the cyclosporin derivative/surfactant/water.

27. The topical scalp and transdermal preparation as set forth in claim 24, wherein a weight ratio of the surfactant to the cyclosporin derivative is 1/100 to 100/1 in a mixture of the cyclosporin derivative/surfactant/water.

28. The topical scalp and transdermal preparation as set forth in claim 24, wherein the mechanical dispersion instrument is selected from the group consisting of a colloid mill, a roll mill, a sonicator, a high-pressure dispersion instrument (microfluidizer, Microfluidics Corp., USA), Ultra Turrax (Janke and Kunkel, Germany), Nanoget (Nanoget Engineering, Germany) and Brogli (Italy) and mechanical dispersion instruments equivalent thereto.

29. The topical scalp and transdermal preparation as set forth in claim 1, wherein the emulsion encapsulating the cyclosporin derivative is prepared by emulsifying the cyclosporin derivative in an oil phase in an aqueous phase containing an emulsifying agent.

30. The topical scalp and transdermal preparation as set forth in claim 29, wherein the oil phase is selected from the group consisting of plant or animal oil such as sweet almond oil, avocado oil, castor oil, olive oil, jojoba oil, sunflower oil, wheat germ oil, sesame oil, ground nut oil, raisin seed oil, sova oil, rape seed oil, safflower oil, coconut oil, corn oil, hazelnut oil, palm oil and apricot-kernel oil, mineral oil such as fluid paraffin, synthetic oil such as caprylic/capric triglycerides and triglycerides (C₁₀-C₁₈), and fatty acid triglyceride.

31. The topical scalp and transdermal preparation as set forth in claim 29, wherein the emulsifying agent is selected from the group consisting of substances which are relatively hydrophilic and have a surface activating ability, including polyvinyl alcohol, gelatin, polysorbate 80, sodium alginate, sodium oleate, methyl cellulose, albumin, sodium dodecyl sulfate, sodium lauryl sulfate, polysorbate 20 and fluroric (F68).

32. The topical scalp and transdermal preparation as set forth in claim 29, wherein a volume ratio of the oil phase to the aqueous phase is 0.01/1 to 1.2/1.

33. A hair growth-promoting composition for use on hair, formulated by applying the topical scalp and transdermal preparation as set forth in claim 1, in which a cyclosporin derivative is encapsulated in a carrier selected from the group consisting of a liposome, microcapsule, microsphere, composite particle and emulsion.
